SEQUENCE LISTING (19 sequences)

SEQ ID NO: 1 - RNA, 1406 nt, Brevibacillus laterosporus
  source 1..1406: mol_type = unassigned RNA
tgcagtcgagcgagggtcttcggaccctagcggcggacgggtgagtaacacgtaggcaacctgcctgtaagactgggataacatagggaaacttatgctaataccggatagggttttgcttcgcctgaagcgaaacggaaagatggcgcaagctatcacttacagatgggcctgcggcgcattagctagttggtgaggtaatggctcaccaaggcaacgatgcgtagccgacctgagagggtgaccggccacactgggactgagacacggcccaaactcctacgggaggcagcagtagggaattttccacaatggacgaaagtctgatggagcaacgccgcgtgaacgatgaaggctttcgggtcgtaaagttctgttgttagggaagaaacagtgctatttaaataaggtagcaccttgacggtacctaacgagaaagccacggctaactacgtgccagcagccgcggtaatacgtaggtggcaagcgttgtccggaattattgggcgtaaagcgcgcgcaggtggctatgtaagtctgatgttaaagcccgaggctcaacctcggttcgcattggaaactgtgtagcttgagtgcaggagaggaaagtggtattccacgtgtagcggtgaaatgcgtagagatgtggaggaacaccagtggcgaaggcgactttctggcctgtaactgacactgaggcgcgaaagcgtggggagcaaacaggattagataccctggtagtccacgccgtaaacgatgagtgctaggtgttaggggtttcaatacccttagtgccgcagctaacgcaataagcactccgcctggggagtacgctcgcaagagtgaaactcaaaggaattgacgggggcccgcacaagcggtggagcatgtggtttaattcgaagcaacgcgaagaaccttaccaggtcttgacatcccactgaccgctctagagatagagcttcccttcggggcagtggtgacaggtggtgcatggttgtcgtcagctcgtgtcgtgagatgttgggttaagtcccgcaacgagcgcaacccttatctttagttgccagcattcagttgggcactctagagagactgccgtcgacaagacggaggaaggcggggatgacgtcaaatcatcatgccccttatgacctgggctacacacgtgctacaatggttggtacaacgggatgctacttcgcgagaagatgctaatctcttaaaaccaatctcagttcggattgtaggctgcaactcgcctacatgaagtcggaatcgctagtaatcgcggatcagcatgccgcggtgaatacgttcccgggccttgtacacaccgcccgtcacaccacgggagtttgcaacacccgaagtcggtgaggtaaccgcaaggagccagccgccga

SEQ ID NO: 2 - DNA, 19 bp, synthetic construct
  misc_feature 1..19: note = synthesized sequence, primer
  source 1..19: mol_type = other DNA
gtgccagcmgccgcggtaa

SEQ ID NO: 3 - DNA, 20 bp, synthetic construct
  misc_feature 1..20: note = synthesized sequence
  source 1..20: mol_type = other DNA
cggttaccttgttacgactt

SEQ ID NO: 4 - DNA, 2484 bp, Brevibacillus laterosporus
  source 1..2484: mol_type = unassigned DNA
atgttcaataaaagccaagtagtttctccaaatgctaatctctttactcaacagctatctaatcaagtatctcagactatggggaggttcggtgataaagctgttgcctttctaaaaaaatggcatattcttacacttactatgggttttttattaggaagagctcttattcttgaagaattatcaccatttgttgttccttacttcatagtgatgtactttctccgtcgggatacattgctagttagtgcattgggaatgttggtaggctcattagctcattcaattcctctaggtatgcagacgttactgagtatcatacttgccgctggtgtgtgccgtatctcggagaaatggaaacgaaaagacttttcctataccccatttatggtggtaggaaccgtatttgctagccatctattatgtaaggtcattcagaatcaggtaaacacatacagtgtgactatgattgcggttgaagcaattcttagttttgttttgacacttatctttattcagtccttatccatcattcacataaataaaccatttgaacctttaaagacagaagaaattatatgccttgtcattttattagcttctttaatgacgggaacagtaggctgggttatacagggggtttccatggaacacatcctgtcccgttatcttctattattattcgcttttgttggaggagggacggtcggggccgcagtgggggtggttaccgggctcattctcagtttggctgatgtgagtgccttacaacaaattagtttactcgctttcgcaggattactagcggggctattaaaggaaggaaagaaaattggtgtagtagcaggactcttaatcggaacttccattctgggaatctatggtggtgccgaaagtggattatatctctctttaatcgagacttcgattgccgctttcctctttttattaacgccagcttttatttggaaaagagtagccagcttcattccggggacttcggaacacatacaatctcaccaggaatatatgcggcgcatacgtgacatgacagcaggtaaaattcaacagttttccgatttgtttattgagttatcccattcattcgctcaaacagctgaaacaaaaaatggagttgaggaggaagagatagatctttatttaagtaaagtgacggagaggacatgccagctttgctggaagaaagagcagtgctgggagcgagatactcaaatgacctacgaagcgatgagtaacatgcttattacggtattcgagcatgggacaatgcaaggagcaactataccaaaggagtgggaaaagaaatgcgtgaaaacagaaaaggtaatccaacatatggagcaagagtaccaccagatgattgaacataatcaattaaaaaaacaaattcaagatagtcgcaagctggtagccgatcaattatcaggagtatcacgggtcatgagcgattttgcacgggaaatccaaagggaaggacttgcactaagcctacaagagaagcaggtttcaaaagcgctggagggtttaggactatccgtaagacgtgtagatattcatagcttagaagaagggaaggtggatatagaaatcagtcagcctagttgttatgggagggatgaatgcgccaagattgtggctccgatgcttaccgagattttaggcgagaacattattgtgaaggaacgacaatgtgaagcctttaaggatcagcattgcaaaatgtgcctggcttcagcaaaaacatatgagatcgaaattggagtggcaggagccgctaagggtggcaaactattgtcaggagatagctttaaaacaatggatttaggaaatggaaaggtggctgtggcgatcagtgatggaatgggaaacggggagcgcgcttatatggaaagtcaatcagcattggacatgctgcaacagctcttaaaatcagggctggatgaaaaactgtctattaaaacagtaaattctgttttggcattacgttcggctgatgagatgtttgcaacagtggatttggctgtaattgacttacagactgcaatgacacggtttattaaaattggttctacaccttcgtttattaaacgaggagatgatgtgtttatggtatcggcaaataatttaccggttggtattttagatgatattgaagtagatattgtgactagaaatctaaaggcaggtgatctattaatcatgatgtcagatggaatttttgaggctccacaccatatcgaaaatagaccggtgtggatgaagcgattaatctctcagcttgaaacagaagaccctcaggaaatctctgacttattattagaaaaagtgattagagaacatagtggtgaaatcgtagacgatatgacggttttagtcgctcgaattgatcggtttgtaccgcaatggtcggcgattcaggtatcaggaatggagaagatggaacggccacgtgtagtaagttaa

SEQ ID NO: 5 - DNA, 987 bp, synthetic construct
  misc_feature 1..987: note = synthesized sequence
  source 1..987: mol_type = other DNA
gatgatccagaatacatcgcagagcttgcacgcaaaaataattttatgtccaagccgggtgagatcatctttttgatccctgataattaagccatttggatctaagctaaaataaatttacgaaaacgtgcaatagattttaaaaacaggaaggtcattttaattaaatggcgaatataatataacaagcccgtttcgccgagcgcgaatcacctgatggccgacgttgaacgcaagacgtcggttttttttattttgattctttttcaatttaaagtgataaagcataaatcttctattcaatcctcgacaggtacctcaatcaattcgtatcgtcttcagcataaatccatgaacgatcaaacaaataaacgagatacgtttaaatttgtcggacgattttaggaatatgcctctatcgtttgacaaaatttacagcagtccattcgtataattgggtacacaaaactcaaaacgggtggtgctagtattattcagtccttatccatcattcacataaataaaccatttgaacctttaaagacagaagaaattatatgccttgtcattttattagcttctttaatgacgggaacagtaggctgggttatacagggggtttccatggaacacatcctgtcccgttatcttctattattattcgcttttgttggaggagggacggtcggggccgcagtgggggtggttaccgggctcattctcagtttggctgatgtgagtgccttacaacaaattagtttactcgctttcgcaggattactagcggggctattaaaggaaggaaagaaaattggtgtagtagcaggactcttaatcggaacttccattctgggaatctatggtggtgccgaaagtggattatatctctctttaatcgagacttcgattgccgctttcctctttttattaacgccagcttttatttggaaaagagtagccagcttcattccggggacttcggaacac

SEQ ID NO: 6 - DNA, 25 bp, synthetic construct
  misc_feature 1..25: note = synthesized sequence
  source 1..25: mol_type = other DNA
ggatgatccagaatacatcgcagag

SEQ ID NO: 7 - DNA, 21 bp, synthetic construct
  misc_feature 1..21: note = synthesized sequence
  source 1..21: mol_type = other DNA
cgctaaaacgtctcagaaacg

SEQ ID NO: 8 - DNA, 5919 bp, Brevibacillus laterosporus
  source 1..5919: mol_type = unassigned DNA
atgagcgataaaatacttgttgtggacgatgaacatgaaattgccgatttggttgagctatacttgaaaaacgagaattatacggttttcaaatactataccgccaaagaagcgttggaatgtatagacaagaccgagcttgaccttgccatattggacatcatgcttcccggcgcaagcggtctcgctatctgccaaaaaataagggacaagcacacctatccaattatcatgctgaccgcgaaagatacggaggtagataaaattacagggctaacaatcggcgcggacgattatataacgaagccctttcgcccgctggagttaattgcgcgggtcaaggcgcagttgcgccgatacaaaaaatacagtggagtaacggagcagaacgaaaatgttatcgtccactccggccttgtcattaatataaacacgcatgagtgttttctgaacgagaagccgttatccctcactcccaccgagttttcaatcctgcgcatcctctgtgaaaacaaggggaatgtggttagctccgagcagttatttcatgagatatggggcgacgaatatttcaacaagagcaacaacaccatcaccgtgcatatccggcatctgcgcgaaaaaatgaacgacaccattgataatccgaagtatataaaaacggtatggggggttggctataaaattgaaaaataagaataaaaaaaccgattatttcaaactaaaaagaaaactctacctgtatatcgttgtgattgttatggcagcagttgtatccgtgttgtttttgcgtttatttatcaaggggacacttggggagtggaccgtacgttttttggaaaacagttatcacttagagcgtcaggacgcgatgataatatatcagtataccatacggaacaatatagaaatctttatttatgtggcaattgccattagtattcttattctatgccgcgtcatgctttcaaaatttgcaaaatactttgacgagataaataccggcattgatatacttattcagaacgaagataaacaaattgagctttctgcggaaatggagttcatggaacagaagctcaacactttaaaacggactctggaaaagcgagagcaggatgcaaagctggccgaacaaagaaaaaatgacgttgttatgtacttggcgcacgatattaaaacgccccttacatccgttatcggttatctgagcctgcttgacgaggcgccagacatgccggtagagcaaaaggcaaagtatgtgcatatcacgttggacaaagcgtatcgcctcgaacagctaatcgacgagttttttgagattacgcggtataacctccaaacgattacgctcacgaaaaagcacatagacctatactatatgctggtgcagatgaccgatgaattttatccccagcttgctgcgaatggaaaacaggcggttatccatgcttccgaggatttgaccgtatccggcgaccccgataagctggcgagggtctttaacaacattttgaaaaacgcggctgcatacagcgaggacaacagcgtcattgacattacggcgggcctctccggggatgtggtgtccatcgctttcaagaacgccgggagcatcccaaaagataagctcgctgccatatttgaaaagttctacaggctggacgatgcccgttcttccgatacgggcggcgcgggacttggattggcgattgcaaaagaaatcattgttcagcatggcgggcagatttacgcggaaagcaatgataactacacgacgtttacggtagagcttccagccttgccagacttggttgataaaaggggttcctaagaaatgaatatcgtttcttaggaaaatctcaaggtcatctttactttttcttaggaaattaacaatttaatattaaaaaacggctcgttcttacgcggtagacttaaaaccgtaagaacgagcttttttcgtttcgtcagagaaagatatgacaagtttaccgcgattggcatccccgttttatttcttattggtgcctttcgcagaaaggaatggtcttaattatgaaaaacatcggcattaccgtttatggatgcgatcaggatgaggcagatgcattccgtgccctttcgtcgcgctttggcgttatgccctcgataattaacgccgacgtgtcggaatccaacgccatatccgcccctttcaatcaatgtatcagtgtgggacataaatcagaggtttccgcgtctattcttcttgcgctgaagagagccggtgtgaaatatatctccacccgaagcatcggctgcaatcatatagatacaactgccgctaagagaatgggcatcactgtcaacaatgtggcgtattcaccggatagcgttgccgattataccatgatgctgatgcttatggcagtacgcaacgcgaaatctattgtgcgctctgtggaaaaacatgattacaggttggacagcgtccgtggcaaggtactgagcgacatgacagtcggcgtggtgggaacgggccatataggcaaagcggttattgagcggctgcggggatttggatgtcaagtgctggcttataatcgcagccaaagtatagaggcaaactatgttccgtttgatgagttgttgcaaaatagcgatatcgttacgcttcatgtgccgctcaatgcggatacgcgccatatcattggccacgaacaaatgaggagaatgaagcaaggcgcgtttattatcaatacagggcgcggtgcgcttatagataccgatgcgctggttaaagcattggaaaacggaaaactgggcggcgccgcattggatgtattggagggagaggaagggcttttctattttgattgcacacaaaaaccaattgataatcaatttttactgaaacttcaaaggatgcagaacgtgataatcacgccgcatacggcttactataccgaacaggcgttgcgtgataccgttgaaaaaacaattaaaaactgtttggattttgaaaggagcttggcacatggatagagtaaaagttgcaatcctgtttggcggttgctcggaggagcatgacgtgtcggtaaaatccgcaatagagatagccgctaacattgacaaagaaaaatacgagccgttatacattggaattacgaaatctggtgtttggaaaatgtgcgaaaaaccgtgcgcggaatgggaaaacgacaattgctgttcagccgtactctcgccggataaaaaaatgcacggtttgcttgttataaagaaccatgaatatgaaatccaccacgttgatgtagcattttcggttttgcacggcaagtcgggtgaagatggatcaatacaaggtctgtttgaattgtccggtatcccctatgtgggctgcgatattcaaagctcagcgatttgtatggacaaatcgctggcatacatcattgcgaaaaacgctggcatagctactcctgaattttgggttattaataaagacgataagccggcgatagatgcgtttacctatcctatttttgttaagccggcgcgttcaggctcatcttatggcgtgaaaaaagtcaatggcgcagacgaattgaacgccgcaattgaatcggcaagacaatatgacagcaaaatcttaattgagcaggctgttttgggctgtgaggtcggttgtgccgtattgggaaacagttccgagttgattgttggcgaggtggaccaaatcaggctgcagcacggaatctttcgtattcatcaggaagccgagccggaaaaaggctctgaaaacgcagttataaccattcccgcagacctgtcggcagaggagcgaggacggatacgggaaacggcaaaaaaaatatataaggcgctcggctgtagaggtctagcccgtgttgatatgtttttacaagataacggccgcattgtgcttaacgaagtcaataccctgcccggtttcacgtcatacagtcgttatccccgtatgatggtcgctgcaggtataacacttcccgaactgattgaccgcctaatcgtattagcgttaaaggggtgataagcatgaaaaaaggatttacttttttagatgaaatattacacggtgttcgatgggacgctaaatatgccacatgggacaatttcaccggaaagccggttgacggatatggagtaaatcgcatggtaggaacatacgagttggccgacgcgcttttgaaggtaaaagaactggctgctactcaagggtacggattgcttctatgggacggttaccgtccccaacgcgctgtaaactgttttttgcaatgggttgcacagccggaaaatgacctgacaaaggaaagatattatcccaatattgaccgaaccgagatggtttcaaaaggatacgtggcttcaaaatcaagccatagccgcggaagtgcaattgatcttacgctttaccgattagacacgggtgagcttgtaccaatggggagcggatttgattttatggatgaacgctctcatcatgcggcaaaaggaatttcaggcaatgaagcgcaaaatcgcagacgtttgcgttccatcatggaaaacagtgggtttgaagcatatagcttcgaatggtggcactatgtattaagaaacgaaccataccccaatagctattttgatttccccgttaaataagcttttaaccgttgcacgggcaacctatataatgctatgtctttcggcacggaaaccgtcgtttgcaactggtttgtagggaatttatatatagtagatagccggcgtcgagatcggaaagacccacgcctacctgatcgccgccgccctcgccaagcgcgggcgcgtgggcgatttctggctcaaatgcaaatatcccggcatggcgtatattgccgagatgcccgtggtggtggccacgtccagcatcgccctgcagaaagctatcgttaaggactacatacccgaaatctcccgcgcgctgacccagcgcggcattatcaccaagccactcacttaaggaggaaactgaaaatgaagaagtggggttttttattgttatttttattgtgcttaggttttgccttcattaataaagcactgttttttcaagataaagtagagattcaaaaatatgatcaaaatcacaaagataatatagataacatagagaatatagggacccccttaagtatccaaaagaaagagattgcaaaagaacagatctatcaaggaaatctgctcttaatcaacagtaaatatcctattcgccaagagagtgtgaaatcagatatcgtaaatttatctaaacataatgaattgataaatggatacgggttgcttaatacgaatatttatctgtcaaaaggaatagcacaaaaattttcagagatgatcaatgatgctgtaaaggaaggggttagtcatttttttattaatagtggctatcgtgactttgatgagcaaagtgtgctttaccaagaaatgggggctgattatgccttgccagcaggttatagtgaacataatttaggcttatcacttgatgtaggatcaagcttaacgaaaatggatcgagcacctgaaggaaagtggctgaaagaaaatgcctggaaatacggctttatattacgctatccaaaggataaaactgatgttactggaattcaatatgaaccatggcatatccgttatgttggtttccctcacagcgcgattatgaaagaaaagaatttcgctctagaagaatatatggattttttgaaggaacagaagtccattacgactacaatagaccaccaagtctataaaatcttttactatcctatctcccaaaatacaacgatccctgtgcctgcgaatgtccagtatgaaatttcaggcaacaatatggatggcgtgattgtgacagtgtattctggtaagcgatactaa

SEQ ID NO: 9 - DNA, 1049 bp, synthetic construct
  source 1..1049: mol_type = other DNA
cttctcaatgagatgctcttcagcatgagcttacatagagagtgaaaattcagtcactattgtttacctataatttgtaagcttgtggggtaaaacgatccgaagatacataattaaattttttttataagaaaattgaagttttttaaagcaggaaaaatggggtataaatgtagtataattataattattctagttgtggggagagaaacgaaatggagcttactggtcaacgcttgttcgtattatggagaaccttttctcaacttgattttctacgtcaaattagaggacgtgacatcccggatcagtctgtgaatgaattaattaacaaagtaaaaaatttagatgaagtggaagtattaccgattgatacagaacaattacgtgaaacggtaatcaaacttcgtaaatagataaaagtaaggatgctctgctgcattctttctatttggggtaccgtcaggaaaatgcggatttacaacgctaagcacatttccaagacgattcccccatttttaacaacggttccgacggaaaaacggagagagtatcaacaacaactagccgaacatagcaaatgtgagtttttagagatgcccataagcgaaccagagtggcttgacaagaaattggaggagaaatattggaaaataaaaaattgacattcgtattattcataatctatgtattagcacttatttggttagttctttttaagttacagttttcttttgatcaaatagacagagtgcgagtaattaatatgattccacttaatgggtctgtcttttctgaggtgtataacaatataaggatttttgtacctttgggtatctatatttgcatgttgaaaagtaagtggtcttttatgaaaaaacttctttcaattattggttttactctggcatttgagactatacaatttgttttagcaatcggaagaagtgatattactgatatactggccaatacacttggaggtacaatcggtattggtatttatgagctattattcaagattctatcgtttctgagacgttttagcg

SEQ ID NO: 10 - DNA, 20 bp, synthetic construct
  source 1..20: mol_type = other DNA
ccgagtttcttgcatagagc

SEQ ID NO: 11 - DNA, 20 bp, synthetic construct
  source 1..20: mol_type = other DNA
agctgtgcccatggattgca

SEQ ID NO: 12 - DNA, 1524 bp, Brevibacillus laterosporus
  source 1..1524: mol_type = unassigned DNA
atgaagaagtggggttttttattgttatttttattgtgcttaggttttgccttcattaatagagcaccgttttttcaggataaagtagaaattcaaaaatatgatcaaaatcacaaagatcatttagataaaagagggacttctgaaagtacccaaacaaaaacgattacaaaagaacagatttatcaaggaaatctgctcttaatcaacagtaaatatcctgttcaacaaaagagtataaaatcggatgtcataaatttatttacgcacaaagaattgacaaagggatatgggttacttgataacgaaattaaattatcagaagaaatagctgaaaattttcggtgatgatagctgcggctgtagaggatggggttagtaatttcttaattagcagtggttatcgagactttgatgagcaaaacagactgtacgaggagatgggggctgactatgctttgccagctggtcatagcgaacacaatttaggattagcacttgatgtagggtctactcaaatgaagatggaagcacctgaaggagagtggatagaagaaaattcttggaaatacggctttatattacgctatcccttggataaaacggatgttacaggaattcaatatgaaccttggcatattcgctatgtcggcttgcctcacagtgcgattatgcaggaaatgaatttacctttggaagaatatttggattatttaaaagaagaaaagagcatattatcccatttctcaaaacgagacaattgaagttgaagtaccagcgaatggacagtatgaaatatctggtaataatattgatggagtgattgtgaccacattttcttgaattaggttctaaaatgataaagtcataggtttaaatgtaaagttaggagacgtattctatatgcttacacttacagttttgtatacttatttttgtactattattttttgtattgtgtttcaaattggatttttagtaaaggcgcaaaaaaatttatctattaagcattttctatgggtgtatgtttttcttttctacctcgcgctagtgtatatggtgacggggatagggaatatatgggtaataggaagatatgaaacattaatacgtataggtgaaatcaacttactcccttttgcttctgaaggcattactacgtatattttgaacattattttatttatgccgttaggttttttattaccaactatttggccgcagtttagatcaattaaaaatgttgcatatactggattatttttttcattggctattgagctaagtcaattgctaaatcataggattacagatattgatgatttatttatgaacaccctaggggcgattattgggtatttaatatatagtgctttatttgtaatggtacataaaagaggtgaaaaaaagcttgatattaaatcctctctagtagtaaaatacgaggctgttttttatttagtttgctcatttataggtatgatattacttcattatccaattttatttcgaaaaattatctaa

SEQ ID NO: 13 - DNA, 1049 bp, synthetic construct
  source 1..1049: mol_type = other DNA
cttctcaatgagatgctcttcagcatggcggtagagatcaggattgtaaggttcattctttttgagaatgttgtagatatgtttcaatttaccgcgatcgacacccctgttttggtgcctttcgcagaaaggaatggtcttaattatgaaaaacatcggcattaccattacacaggcaaaatctcaaatattgagccggttagggggaagattagagaaatatatcatacatccaactacattgcaggtgaattttcataagtggtacccatgctgatttaacgaggctgaaccgcctattgacaatgggcggagcaaacgggcggtgtggagtatcgcccatctattcgttcagtcttgtttttcaggctcatcttgccgcaggccagcaattcgcagatcaattgctatagttctgctgggtaccgggaggaacaggccgcccttgccggccatatcctggaggcgctctgcagctgcagcctttctctgtccgtggccggcgtcgagaaaggaggaaactgaaaatgaacattatgattgtttgtatatataacgagaaaaggggttaggttatcagctgcctgagggaacaaaaattatttgacaagagagaggttgatagtatgaaaaatatcggcattaccatttatggatgtgagagggacgaggctgaagtgttcaatgaactttcgccatgctttggtgtcatacctgccattacgagcacttgatattggttacggttatacagctgatggggggaaaacattcccgtttcgtggtaaagggattaatcttatgccaacacttgatgaagtacttaaccattttccagatcgttcttttcttattcatattaaaagtgatgatgaaaatgaaggaatccaattggctactcacctcaagaaattacccgcgaaacgccttgatcaactaactgtatctagtggcgataaaccaattgctgcgataaaagagcgcatccctagtttacgaaacaatgtcaaaggcaaccatgaaaaagtcgtttctgagacgttttagcg

SEQ ID NO: 14 - DNA, 20 bp, synthetic construct
  source 1..20: mol_type = other DNA
cggcaaagacactgaacggc

SEQ ID NO: 15 - DNA, 21 bp, synthetic construct
  source 1..21: mol_type = other DNA
ggtggaatttggccaaatcgc

SEQ ID NO: 16 - DNA, 1814 bp, Brevibacillus laterosporus
  source 1..1814: mol_type = unassigned DNA
ttacacgctaattcgattcatggccttagtacgctttggatttacatcataatcatgtaatccttttttatgtctagctagcctctgaaagtaggaaaggtactgcttttccaatggatacttcccttttctagtggctaatactagctgatggatgcaatgtagttcctccaattgaaacttttatttcattagataatggaacattgtattcatcaataattgttctccaccattcccatgctagacctgtgcattctttggccaaaattcttatatttttagaattaggtggaagtggtattacggtagagaaatgagctgttctgtttttgtagtttccatcccaagttttgtgggtaagtacctcatttccatcttcatcgtatgaaaattcatcccaggatacttcaaattgagcaacgtatgcaccatgatgatcaagaatgattttacctttagaatattctgtagaggtagtctcaatatagtctgtattgttatgaatagcagcaattgaattatcttttaagaaaacactggtatatgaaatagggtatgctggattttttagactaaattccgcattatctttaataatatttcgtatttcatcaaagtcttttgtaacaaccttgttatgctcttttgaatctccccctaatactacagcagtaaaggagctatcttcaaaaatgtctttgtactgtccgctagcttctacattggtattcttgagtaaggctttaaatgcagcttgtacatctttgctcttagaggttgtttctaattttacataaatggttcgcccataagctacatttgacaccataacaggtggagcttcatcacttacccctttacgggtcagttccttaaaagaaacactatcatcaaagaggtctgatggattgtctggcagctctgcatttaccgtataaaatatttgcttatacgcagcaatcatcactttcttttctccgctagcaatagcattaaagtcaattcccagtgaattgttaagaattttagagttaacattaagagcacttgctatttgatttttgctataaaccatggattctgcatattgtagtcttgcaggtaaagtatttgatgttgaatacttttcactccaagtagatactaattcatcaattgctccagacacattaccatatgttgggttttcaacgattattgaattttctcttttcatcccaggtaaatctatactaatattcaagggctttctcttggccgttaataaagtaggttgatttgctacaaaagcgtcgtttgcaagttgtattgcgcctggataggtacgattcgccacagaatcaataatagaaatatccactggtgaagttgtaagtgattttttctcacgtttaaccactataaatttaccatttgatttaacaccttctgttggaacataactatttatttgatcaccatttgttgctaaaatatcgtgaacatcatagtttaagttagctattccagtatcaatgtcattaggctggtttgtttcactagctacactcgtattagatttttcagcaaaggaaataaaaggataactaataatgcataaacttgtgcttaagcaaatgagtaattttgttgcttttaaacggtttagaattctcaagaaacatcaactccttttggtaaatcataccgtctatttttggtatttagtatagttccagagactcaactagttagtaatatattattatgaactatataaagaaagtgtccgaaggaagattaaaagtaaaaaggaccataataattcattt

SEQ ID NO: 17 - DNA, 1049 bp, synthetic construct
  source 1..1049: mol_type = other DNA
cttctcaatgagatgctcttcagcatgaagtaattgatccagttatccacgaagatatagttgatatggtgcataaccaagtatcctttatgaaacagataaaggagttgctagataaggaaagtctacaaaagataaaagatcagtcaagttattataaggatatttataaggcagagagagaattaaatttggacattccaaaaggaaaggatggactaacaaaatataaatcttctttagagcaaatgaattcattactaacaaaaagcatagttgaggattataaaaaatgattgagaaaattatgttcttaaccacagaagttgacgttaaaagtaacggaagtttaccgagtttcttcattaaaatagatgtttaggaagcgactattatgaagaaaaaaaacgtgttttttttaatattgagtattttttcgtgcttgttggagagaaaataaggataaagaaaacgactaaagtccttgtgtagcaaggattttagccgtttgaagagtgttgaccaattaaaaatagtgctttagttgtatcttcctaggctaatcagtgaaggaattaactagcgagtaagcgtatcttaattgtcgattttgattactctagtcatgtggagagtgtggctttgcttgataaaaaaggaacaggtatcttaaaacattgatataacagtgatttttgctatataacagtgattcgtattttagtgtaacagatatcgaaataatggattgttgacgaactgttgaccaaattcataaaactatatcacaaaagagtaaaagacaccggataattgatctggtgtcttattttgtggtgccagatgctttgggttgaaatatggattcaaatttattagcagcttcttcatctgctgattggagggcgtgtccgtagacattcatcgtggtagaaatacttgggtgaccaagcctttcagaaattatcttggcgtgtactccttgattaattagaatcgttgcagaagtaagacgcaaatcatggaatcgtttctgagacgttttagcg

SEQ ID NO: 18 - DNA, 22 bp, synthetic construct
  source 1..22: mol_type = other DNA
ggtgtcaatgattgtttcgagc

SEQ ID NO: 19 - DNA, 21 bp, synthetic construct
  source 1..21: mol_type = other DNA
gtctccctgattcagtctgtc

(19) United States
(12) Patent Application Publication
(10) Pub. No.: US 2025/0281388 A1
(43) Pub. Date: Sep. 11, 2025

(54) COMPOSITIONS COMPRISING BREVIBACILLA MULTIFUNCTIONAL FERMENTATES AND METHODS OF USE

(71) Applicant: Nutrition & Biosciences USA 4, Inc., Wilmington, DE (US)

(72) Inventors: Perry Caimi, Kennett Square, PA (US); Ziang Li, Newark, DE (US); Christine McInnis, West Chester, PA (US)

(21) Appl. No.: 18/862,576

(22) PCT Filed: Mar. 16, 2023
(86) PCT No.: PCT/US2023/064499

§ 371 Date: Nov. 4, 2024

Related U.S. Application Data
(60) Provisional application No. 63/341,493, filed on May 13, 2022.

(51) Int. Cl. A61K 8/99; A61Q 19/00

(52) U.S. Cl. CPC A61K 8/99; A61Q 19/00

(57) ABSTRACT

The present disclosure is directed towards compositions comprising Brevibacilla multifunctional fermentates and methods for providing multiple functions to compositions. More specifically, the present disclosure is directed towards compositions comprising at least one Brevibacillus laterosporus multifunctional fermentate, or a fraction thereof, and methods for providing at least two functions to a composition comprising a preservative.

### CROSS REFERENCE OF RELATED APPLICATIONS

This application is a 371 of International Application No. PCT/US2023/064499, filed Mar. 16, 2023 and claims the benefit of U.S. Provisional Application No. 63/341,493, filed May 13, 2022, the entire content of each are hereby incorporated by reference.

### FIELD OF THE DISCLOSURE

The present disclosure is directed towards compositions comprising Brevibacilla multifunctional fermentates and methods for providing multiple functions to compositions. More specifically, the present disclosure is directed towards compositions comprising at least one Brevibacillus laterosporus multifunctional fermentate, or fraction thereof, and methods for providing at least two functions to a composition comprising a preservative.

### REFERENCE TO SEQUENCE LISTING SUBMITTED ELECTRONICALLY

The content of the sequence listing electronically submitted with the application as an XML file (20230314_NB41887PCT_SequenceListing; Size: 36740 bytes; Created: Mar. 14, 2023) forms part of the application and is hereby incorporated herein by reference in its entirety.

### BACKGROUND

Preservatives are added to products such as food products, beverages, pharmaceutical drugs, paints, biological samples, cosmetics, personal care products, and many other products to prevent decomposition by microbial growth or by undesirable chemical changes.

Traditionally, water-based home and personal care products use broad-spectrum preservatives, such as isothiazolinones and formaldehyde releasers, which may cause skin sensitization. As such, these traditional preservatives (isothiazolones, formaldehyde releasers, parabens) are under pressure from regulatory agencies, environmental working groups, and social media. Many personal care manufacturers have turned to “soft” preservatives such as phenoxyethanol and organic acids (such as benzoic acid, sorbic acid or citric acid).

Soft preservatives usually require high use levels for efficacy, and some have limited preservation efficacy (e.g. phenoxyethanol is weak against fungi) or have limited pH range efficacy (organic acids). Since these soft preservatives alone do not provide full spectrum efficiency, formulators use them in combination with preservative enhancers such as sorbitan caprylate, caprylyl glycol, ethylhexyl glycerin, 1,2,-hexanediol and other chemical boosters to provide full protection to the formulation (Reich M., 2018, Chemical and Engineering News, volume 96, issue 39). A preservative enhancer such as sorbitan caprylate can act like a surfactant to crack open bacteria and cell membranes, enabling the preservatives to penetrate the microorganisms and inhibit them. Formulating blends of soft preservatives with preservative enhancers can be complicated. Traditional preservatives such as parabens had minimal impact on formulation properties, while cosmetic makers typically need to add a higher percentage of the alternative blends to do the work of a traditional preservative.

Even with these options today, formulators, such as but not limited to personal care formulators, still experience difficulties in compatibility and preservation across their diverse products and formulations.

Surfactants are compounds that reduce the surface tension between the liquid and air interface (Berg, J. C., 2010, World Scientific., pp. 137-147). Surfactants are widely used in personal care products and cosmetics because of their amphiphilic nature, i.e., they are compatible with both water and oil. Because most leave-on formulations in the personal care industry (e.g., cream, lotion, serum, sunscreen, conditioner, etc.) are oil-in-water emulsions, surfactants are important ingredients to stabilize the formulations for better shelf-life stability. For rinse-off formulations like cleanser or body wash, surfactants can form micelles to create foams and remove soils with its amphiphilic property.

Cosmetics and personal care product often use antioxidants to defend skin against environmental stress caused by free radicals. Antioxidants can reduce or remove the free radicals from the oxidation reactions, and therefore, prevent the skin from wrinkling, photoaging, elastosis, drying, and pigmentation (Kusumawati, I. and Indrayanto, G., 2013. In Studies in natural products chemistry. Vol. 40, pp. 485-505. Elsevier). Currently, there is a great interest for formulators to find natural-resource-based surfactants and antioxidants for replacing use of synthetic substances.

Taken together, there remains a need to find methods and compositions for providing multiple functions, wherein such multiple functions include but are not limited to an antioxidant, a surfactant, a foam stabilizer, a foam enhancer, an enhancer of preservation efficacy, or any combination thereof.

### SUMMARY

The present disclosure is directed towards compositions comprising Brevibacilla multifunctional fermentates and methods for providing multiple functions to compositions. More specifically, the present disclosure is directed towards compositions comprising at least one Brevibacillus laterosporus multifunctional fermentate, or a fraction thereof, and methods for providing at least two functions to a composition comprising a preservative.

The present invention is based on the discovery that a fermentate from Brevibacillus laterosporus has multiple functions, such as enhancing the efficacy of a preservative while simultaneously acting as an antioxidant, a surfactant, a foam stabilizer, a foam enhancer, and/or a UV light protector.

In one aspect, the inventors have unexpectedly observed that a fermentate Brevibacillus laterosporus enhances the efficacy of a preservative in a composition such as a personal care composition. Furthermore, the inventors have unexpectedly observed that while the fermentate of Brevibacillus laterosporus enhances the efficacy of a preservative in a composition such as a personal care composition, it can simultaneously function as an antioxidant, a UV light protector a surfactant, a foam stabilizer, and/or a foam enhancer in said composition.

In one embodiment, the composition is a composition comprising (a) an effective amount of a Brevibacillus laterosporus multifunctional fermentate, or fraction thereof, and (b) one or more preservative wherein each of (a) and (b) is present in the range of 0.001 to 10% by weight of the total composition.

In one embodiment, the composition is a composition comprising (a) an effective amount of a Brevibacillus laterosporus multifunctional fermentate, or fraction thereof, and (b) one or more preservative wherein each of (a) and (b) is present in the range of 0.001 to 10% by weight of the total composition, and, wherein the multifunctional fermentate enhances said preservative efficacy.

In one embodiment, the composition is a composition comprising (a) an effective amount of a Brevibacillus laterosporus multifunctional fermentate, or fraction thereof, and (b) one or more preservative wherein each of (a) and (b) is present in the range of 0.001 to 5% by weight of the total composition, and, wherein the multifunctional fermentate, or fraction thereof, has at least two functions selected from the group consisting of an antioxidant, a surfactant, a foam stabilizer, a foam enhancer, a UB blocker, a blue light blocker, an enhancer of one or more preservatives, and any one combination thereof.

In one embodiment, the method is a method for enhancing the efficacy of a preservative in a composition, the method comprising adding an effective amount of a Brevibacillus laterosporus multifunctional fermentate, or fraction thereof, to a composition comprising a preservative, wherein each of said preservative and said fermentate are present in the range of 0.001% to 10.0% by weight of the total composition, wherein the multifunctional fermentate enhances said preservative efficacy.

In one embodiment, the method is a method for enhancing the efficacy of a preservative in a composition while simultaneously providing an additional function to said composition, the method comprising adding an effective amount of a Brevibacillus laterosporus multifunctional fermentate, or fraction thereof, to a composition comprising a preservative, wherein each of said preservative and said fermentate are present in the range of 0.001% to 10.0% by weight of the total composition, wherein the multifunctional fermentate enhances said preservative efficacy while simultaneously providing an additional function selected form the group consisting of an antioxidant agent, a surfactant, a foam stabilizer, a foam enhancer, a UV blocker, or any one combination thereof.

### DETAILED DESCRIPTION

The features and advantages of the present disclosure will be more readily understood, by those of ordinary skill in the art from reading the following detailed description. It is to be appreciated that certain features of the disclosure, which are, for clarity, described above and below in the context of separate embodiments, may also be provided in combination in a single element. Conversely, various features of the disclosure that are, for brevity, described in the context of a single embodiment, may also be provided separately or in any sub-combination. It will be understood that in the following, embodiments referred to in relation to one broad aspect of the invention are equally applicable to each of the other broad aspects of the present invention described above. It will be further understood that, unless the context dictates otherwise, the embodiments described below may be combined.

Microorganisms, Fermentates, and Fractions thereof.

As used herein, “microorganism” or “microbe” refers to a bacterium, a fungus, a virus, a protozoan, archaea, and other microbes or microscopic organisms.

In some embodiments, the microorganism(s) suitable for use in the present invention can be subjected to treatments that render them non-replicating, for example, exposure to heat, desiccation, γ-irradiation, or UV-irradiation. A non-replicating microorganism(s) suitable for use in the present invention can be a dead cell or a living cell that has been rendered incapable of cell division. A non-replicating microorganism(s) suitable for use in the present invention can be an intact cell or a cell that has undergone partial or complete lysis. In some embodiments, the non-replicating cells can include a mixture of intact and lysed cells.

The microorganism(s) suitable for use in the present invention may be included in a composition according to the invention in live, semi-active or inactivated or dead form. For the purposes of the invention, an “inactivated” or “dead” microorganism is a microorganism that is no longer capable of forming colonies in cultures. The dead or inactivated microorganisms may have intact or broken cell membranes. The dead or inactivated microorganisms may be obtained via any method known to those skilled in the art.

The microorganisms suitable for use in the present invention includes a Brevibacillus laterosporus having a 16S ribosomal RNA sequence displaying at least 97.0% sequence similarity to a 16S ribosomal RNA sequence of Brevibacillus laterosporus strain G2 (SEQ ID NO: 1) deposited at Westerdijk Fungal Biodiversity Institute (WFDB) under number CBS149108. The phylogenetic identity of Brevibacillus laterosporus was determined by sequencing the 16S region with primer set A (5′-GTGCCAGCMGCCGCGGTAA-3, SEQ ID NO: 2) and primer set B (5′-CGGTTACCTTGTTACGACTT, SEQ ID NO: 3).

The 16S ribosomal RNA sequence of Brevibacillus laterosporus G2 is as follows:

(SEQ ID NO: 1)
UGCAGUCGAGCGAGGGUCUUCGGACCCUAGCGGCGGACGGGUGAGU
AACACGUAGGCAACCUGCCUGUAAGACUGGGAUAACAUAGGGAAA
CUUAUGCUAAUACCGGAUAGGGUUUUGCUUCGCCUGAAGCGAAAC
GGAAAGAUGGCGCAAGCUAUCACUUACAGAUGGGCCUGCGGCGCA
UUAGCUAGUUGGUGAGGUAAUGGCUCACCAAGGCAACGAUGCGUA
GCCGACCUGAGAGGGUGACCGGCCACACUGGGACUGAGACACGGC
CCAAACUCCUACGGGAGGCAGCAGUAGGGAAUUUUCCACAAUGGA
CGAAAGUCUGAUGGAGCAACGCCGCGUGAACGAUGAAGGCUUUCG
GGUCGUAAAGUUCUGUUGUUAGGGAAGAAACAGUGCUAUUUAAAU
AAGGUAGCACCUUGACGGUACCUAACGAGAAAGCCACGGCUAACU
ACGUGCCAGCAGCCGCGGUAAUACGUAGGUGGCAAGCGUUGUCCG
GAAUUAUUGGGCGUAAAGCGCGCGCAGGUGGCUAUGUAAGUCUGA
UGUUAAAGCCCGAGGCUCAACCUCGGUUCGCAUUGGAAACUGUGU
AGCUUGAGUGCAGGAGAGGAAAGUGGUAUUCCACGUGUAGCGGUG
AAAUGCGUAGAGAUGUGGAGGAACACCAGUGGCGAAGGCGACUUU
CUGGCCUGUAACUGACACUGAGGCGCGAAAGCGUGGGGAGCAAAC
AGGAUUAGAUACCCUGGUAGUCCACGCCGUAAACGAUGAGUGCUA
GGUGUUAGGGGUUUCAAUACCCUUAGUGCCGCAGCUAACGCAAUA
AGCACUCCGCCUGGGGAGUACGCUCGCAAGAGUGAAACUCAAAGG
AAUUGACGGGGGCCCGCACAAGCGGUGGAGCAUGUGGUUUAAUUC
GAAGCAACGCGAAGAACCUUACCAGGUCUUGACAUCCCACUGACC
GCUCUAGAGAUAGAGCUUCCCUUCGGGGCAGUGGUGACAGGUGGU
GCAUGGUUGUCGUCAGCUCGUGUCGUGAGAUGUUGGGUUAAGUCC
CGCAACGAGCGCAACCCUUAUCUUUAGUUGCCAGCAUUCAGUUGG
GCACUCUAGAGAGACUGCCGUCGACAAGACGGAGGAAGGCGGGGA
UGACGUCAAAUCAUCAUGCCCCUUAUGACCUGGGCUACACACGUG
CUACAAUGGUUGGUACAACGGGAUGCUACUUCGCGAGAAGAUGCU
AAUCUCUUAAAACCAAUCUCAGUUCGGAUUGUAGGCUGCAACUCG
CCUACAUGAAGUCGGAAUCGCUAGUAAUCGCGGAUCAGCAUGCCG
CGGUGAAUACGUUCCCGGGCCUUGUACACACCGCCCGUCACACCA
CGGGAGUUUGCAACACCCGAAGUCGGUGAGGUAACCGCAAGGAGC
CAGCCGCCGA.

The microorganisms suitable for use in the present invention includes, but are not limited to, a Brevibacillus laterosporus having a 16S ribosomal RNA sequence displaying at least 97.0% sequence similarity to a 16S ribosomal RNA sequence of Brevibacillus laterosporus strain G2 (SEQ ID NO: 1) deposited at Westerdijk Fungal Biodiversity Institute (WFDB) under number CBS149108; a Brevibacillus laterosporus having a 16S ribosomal RNA sequence displaying at least 97.0% sequence similarity to a 16S ribosomal RNA sequence of non-sporulating Brevibacillus laterosporus strain A8.11 derived from Brevibacillus laterosporus (SEQ ID NO: 1) deposited at Westerdijk Fungal Biodiversity Institute (WFDB) under number CBS149109; a Brevibacillus laterosporus suitable for use in the present invention includes, but is not limited to, a Brevibacillus laterosporus having a 16S ribosomal RNA sequence displaying at least 97.0% sequence similarity to a 16S ribosomal RNA sequence of Brevibacillus laterosporus ALS311 (SEQ ID NO: 1) deposited at Westerdijk Fungal Biodiversity Institute (WFDB) under number CBS149785; a Brevibacillus laterosporus having a 16S ribosomal RNA sequence displaying at least 97.0% sequence similarity to a 16S ribosomal RNA sequence of Brevibacillus laterosporus ALS317 (SEQ ID NO: 1) deposited at Westerdijk Fungal Biodiversity Institute (WFDB) under number CBS149786; and a Brevibacillus laterosporus having a 16S ribosomal RNA sequence displaying at least 97.0% sequence similarity to a 16S ribosomal RNA sequence of Brevibacillus laterosporus ALS321 (SEQ ID NO: 1) deposited at Westerdijk Fungal Biodiversity Institute (WFDB) under number CBS149788.

In one aspect of the invention, fermentates are provided.

As used herein, the term “fermentate” is to be understood as a composition (complex mixture) produced by propagating living microorganisms (microbial strains) in a nutrient medium. The fermentate may include a cellular mass component from said microorganisms, unspent media components, and metabolites (i.e., unused substrates and/or fermentation end-products). As used herein, a “cellular mass component” refers to any mixture of proteins, lipids (i.e., membranes), carbohydrates, exopolysaccharides, metabolites, etc. from the propagated microorganism. For example, as a microorganism grows it produces new cells that generally include additional cellular mass such as, without limitation, cell membranes, nucleic acids (i.e., DNA and/or RNA) internal subcellular structures, and proteins (i.e., membrane-bound, secreted, and/or intracellular).

Fermentates, fermentate supernatants, or fractions thereof for use in the present invention include fermentates, fermentate supernatants, or fractions thereof from the microorganism Brevibacillus laterosporus.

In one aspect the fermentates, fermentate supernatants, or fractions thereof for use in the present invention includes fermentates, fermentate supernatants, or fractions thereof from the microorganism Brevibacillus laterosporus, wherein said Brevibacillus laterosporus is selected from the group consisting of a Brevibacillus laterosporus having a 16S ribosomal RNA sequence displaying at least 97.0% sequence similarity to a 16S ribosomal RNA sequence of Brevibacillus laterosporus strain G2 (SEQ ID NO: 1) deposited at Westerdijk Fungal Biodiversity Institute (WFDB) under number CBS149108; a Brevibacillus laterosporus having a 16S ribosomal RNA sequence displaying at least 97.0% sequence similarity to a 16S ribosomal RNA sequence of non-sporulating Brevibacillus laterosporus strain A8.11 derived from Brevibacillus laterosporus (SEQ ID NO: 1) deposited at Westerdijk Fungal Biodiversity Institute (WFDB) under number CBS149109; a Brevibacillus laterosporus suitable for use in the present invention includes, but is not limited to, a Brevibacillus laterosporus having a 16S ribosomal RNA sequence displaying at least 97.0% sequence similarity to a 16S ribosomal RNA sequence of Brevibacillus laterosporus ALS311 (SEQ ID NO: 1) deposited at Westerdijk Fungal Biodiversity Institute (WFDB) under number CBS149785; a Brevibacillus laterosporus having a 16S ribosomal RNA sequence displaying at least 97.0% sequence similarity to a 16S ribosomal RNA sequence of Brevibacillus laterosporus ALS317 (SEQ ID NO: 1) deposited at Westerdijk Fungal Biodiversity Institute (WFDB) under number CBS149786; and a Brevibacillus laterosporus having a 16S ribosomal RNA sequence displaying at least 97.0% sequence similarity to a 16S ribosomal RNA sequence of Brevibacillus laterosporus ALS321 (SEQ ID NO: 1) deposited at Westerdijk Fungal Biodiversity Institute (WFDB) under number CBS149788.

It will be apparent that the fermentate may be used directly in the compositions and methods of the present invention, or that one or more fractions of said fermentate comprising active ingredients may be isolated form the fermentate by any suitable means prior to use.

Fermentates can be further concentrated prior to be included in a composition to obtain an effective amount of actives in said fermentate. Fermentates can be spray-dried or lyophilized prior to be include in a composition.

In one aspect the fermentate is a fermentate supernatant. As used herein, a “fermentate supernatant” or “cell free supernatant”, “fermentation supernatant” or “fermentate filtrate” are used interchangeably and refer to a fermentate that is substantially free of viable cells, such as a supernatant of a cell culture of at least one microorganism from which the cells have been removed. It is understood that cells can be removed from the cell culture by any method known in the art and that such removal of cells (such as through centrifugation, filtration) may still result in cell free supernatants that can comprise a trace amount of cells or cell debris. factions thereof, from which all or substantially all, of the cells have been removed. Methods for separating cells from growth media are well known in the art and can rely upon physical methods, for example, centrifugation to produce a cell pellet and a culture supernatant, filtration, ultrafiltration, tangential flow-filtration, normal flow filtration or reverse osmosis. Alternatively, or in addition, the separation method can be ligand-based and include, for example, an antibody that specifically binds to Brevibacillus laterosporus. The antibody can be coupled to a solid support such as a magnetic bead.

In one embodiment the fermentate supernatant is obtained by filtration or centrifugation of the culture medium in which Brevibacillus laterosporus cells were cultivated.

In another embodiment, the fermentate supernatant is obtained by culturing Brevibacillus laterosporus cells in a nutrient medium to obtain a fermentate (whole broth including cells and medium) and adjusting the pH of the fermentate (total fermentation broth) to a pH 2.0-4.0 prior to pelleting out the insoluble cellular matter, and optionally filtering supernatant through a 0.2 μM filter, to yield the cell free supernatant (fermentate supernatant).

In one embodiment the fermentate for use in the present invention comprises Brevibacillus laterosporus fermentate consisting essentially of cell free fermentate. The term “consisting essentially of” in the context of the fermentate includes that at least 90% of the fermentate have the indicated property (e.g. being cell free fermentate). Suitably at least 95% have the indicated property. Suitably at least 97% have the indicated property. Suitably at least 99% have the indicated property. In some embodiments at least 100% have the indicated property.

In one embodiment, the fermentate for use in the compositions and methods and/or uses of the present invention may be substantially free of viable Brevibacillus laterosporus cells, typically containing zero (or substantially zero) viable cells/mL fermentate.

In one aspect the fermentate, and/or fermentate fraction, and/or fermentate filtrate may comprise one or more metabolites, such as but not limiting to soluble metabolites, that were produced during the fermentation of Brevibacillus laterosporus.

In one embodiment a fermentate originating from the culture (fermentation) of Brevibacillus laterosporus may be used in the compositions, methods and/or uses of the present invention.

The growth medium used for preparing the fermentate is any medium comprising necessary nutrients suitable for propagating the microorganism(s) suitable for use in the present invention. Suitable nutrients include but are not limited to amino peptides, peptides, yeast extract, salts, sugars, carbohydrates and/or vitamins. The medium can be based on dairy products, such as milk, cereals, fruits and/or vegetables.

It will be apparent that fractions of the microorganism(s) suitable for use in the present invention, and/or fractions of fermentates, originating thereof, suitable for use in the present invention, may be used directly in the compositions and methods of the present invention, or that one or more of the actives (personal care benefit agents) may be isolated from said fractions by any suitable means prior to use.

As used herein, the term “multifunctional fermentate” refers to a fermentate, fermentate supernatant or fraction thereof, that has at least two activities (functions) selected from the group consisting of an antioxidant agent, an emulsifier, an enhancer of one or more soft preservatives, and any one combination thereof. In one embodiment the multifunctional fermentate originates from the culture (fermentation) of Brevibacillus laterosporus which can be used in the compositions, methods and/or uses of the present invention.

In one aspect the multifunctional fermentates, multifunctional fermentate supernatants or fractions thereof for use in the present invention includes fermentates, multifunctional fermentates or fractions thereof from the microorganism Brevibacillus laterosporus, wherein said Brevibacillus laterosporus is selected from the group consisting of a Brevibacillus laterosporus having a 16S ribosomal RNA sequence displaying at least 97.0% sequence similarity to a 16S ribosomal RNA sequence of Brevibacillus laterosporus strain G2 (SEQ ID NO: 1) deposited at Westerdijk Fungal Biodiversity Institute (WFDB) under number CBS149108; a Brevibacillus laterosporus having a 16S ribosomal RNA sequence displaying at least 97.0% sequence similarity to a 16S ribosomal RNA sequence of non-sporulating Brevibacillus laterosporus strain A8.11 derived from Brevibacillus laterosporus (SEQ ID NO: 1) deposited at Westerdijk Fungal Biodiversity Institute (WFDB) under number CBS149109; a Brevibacillus laterosporus suitable for use in the present invention includes, but is not limited to, a Brevibacillus laterosporus having a 16S ribosomal RNA sequence displaying at least 97.0% sequence similarity to a 16S ribosomal RNA sequence of Brevibacillus laterosporus ALS311 (SEQ ID NO: 1) deposited at Westerdijk Fungal Biodiversity Institute (WFDB) under number CBS149785; a Brevibacillus laterosporus having a 16S ribosomal RNA sequence displaying at least 97.0% sequence similarity to a 16S ribosomal RNA sequence of Brevibacillus laterosporus ALS317 (SEQ ID NO: 1) deposited at Westerdijk Fungal Biodiversity Institute (WFDB) under number CBS149786; and a Brevibacillus laterosporus having a 16S ribosomal RNA sequence displaying at least 97.0% sequence similarity to a 16S ribosomal RNA sequence of Brevibacillus laterosporus ALS321 (SEQ ID NO: 1) deposited at Westerdijk Fungal Biodiversity Institute (WFDB) under number CBS149788.

### Compositions Comprising Multifunctional Fermentates

The multifunctional fermentate originating from the culture (fermentation) of Brevibacillus laterosporus described herein can be used in the compositions, such as compositions that comprise preservatives such as food products, beverages, pharmaceutical drugs, paints, biological samples, cosmetics, personal care products, and many other products.

Preservatives are added in personal care products, preventing growth of harmful bacteria, yeasts and fungi that may cause the product to develop off colors, odors or become unusable due to safety concerns. Contaminants may be introduced during manufacture, shipment, or consumer use. Preservatives are an essential ingredient designed to inhibit growth of microorganisms throughout useful life of a product.

As used herein, the term “preservative” refers to a substance or a chemical that is added to compositions such as food products, beverages, pharmaceutical drugs, paints, biological samples, cosmetics, personal care compositions, and many other products to prevent decomposition by microbial growth or by undesirable chemical changes.

Traditionally, water-based home and personal care products use broad-spectrum preservatives, such as isothiazolinones, formaldehyde releasers and parabens which may cause skin sensitization. Traditional preservatives such as methylchlorosiothiazolinone/Methylisiothiazolinone (CMIT/MIT), Methylisiothiazolinone (MIT), Bromo-2-Nitropropane-1,3-Diol (Bronopol) can cause allergic skin irritation resulting in some social concerns of using these in personal care products (Park, J. et al, 2018, Toxicological Research, 34 (4), 355-361).

As such, these traditional preservatives (isothiazolones, formaldehyde releasers, parabens) are under pressure from regulatory agencies, environmental working groups, and social media. Many personal care manufacturers have turned to “soft” preservatives such as phenoxyethanol and organic acids (such as benzoic acid, sorbic acid or citric acid) (DISADVANTAGES, P. A. (2002). FREQUENCY OF PRESERVATIVE USE. Industrial Biocides: Selection and Application, 270, 14).

Soft preservatives include, but are not limited to phenoxyethanol, benzoic acid, sorbic acid, citric acid, sodium benzoate, potassium sorbate, phenyl ethyl alcohol, lauryl ethyl arginate (LAE) and any combination thereof.

Soft preservatives usually require high use levels for efficacy, and some have limited preservation efficacy (e.g. phenoxyethanol is weak against fungi) or have limited pH range efficacy (organic acids). Since these soft preservatives alone do not provide full spectrum efficiency, formulators use them in combination with preservative enhancers such as sorbitan caprylate, caprylyl glycol, ethylhexyl glycerin, 1,2,-hexanediol and other chemical boosters to provide full protection to the formulation (Reich M., 2018, Chemical and Engineering News, volume 96, issue 39). A preservative enhancer such as sorbitan caprylate can act like a surfactant to crack open bacteria and cell membranes, enabling the preservatives to penetrate the microorganisms and inhibit them. Formulating blends of soft preservatives with preservative enhancers can be tricky. Traditional preservatives such as parabens had minimal impact on formulation properties, while cosmetic makers typically need to add a higher percentage of the alternative blends to do the work of a traditional preservative.

In one aspect the composition is a composition comprising an effective amount of a Brevibacillus laterosporus multifunctional fermentate, or fraction thereof, and one or more preservative wherein each of (a) and (b) is present in the range of 0.001 to 5% by weight of the total composition.

In one embodiment the composition is a composition comprising an effective amount of a Brevibacillus laterosporus multifunctional fermentate, or fraction thereof, and one or more preservative wherein each of (a) and (b) is present in the range of 0.001 to 5% by weight of the total composition, and, wherein the multifunctional fermentate enhances said preservative efficacy.

In one embodiment, the composition is a composition comprising (a) an effective amount of a Brevibacillus laterosporus multifunctional fermentate, or fraction thereof, and (b) one or more preservative wherein each of (a) and (b) is present in the range of 0.001 to 5% by weight of the total composition, and, wherein the multifunctional fermentate enhances said preservative efficacy.

In one embodiment, the composition is a composition comprising (a) an effective amount of a Brevibacillus laterosporus multifunctional fermentate, or fraction thereof, and (b) one or more preservative wherein each of (a) and (b) is present in the range of 0.001 to 5% by weight of the total composition, and, wherein the multifunctional fermentate, or fraction thereof, has at least two functions selected from the group consisting of an antioxidant, a surfactant, a foam stabilizer, a foam enhancer, a UB blocker, a blue light blocker, an enhancer of one or more preservatives, and any one combination thereof.

In some embodiments, the compositions of the invention can include fermentate of Brevibacillus laterosporus, or factions thereof, from which all or substantially all, of the cells have been removed. Methods for separating cells from growth media are well known in the art and can rely upon physical methods, for example, centrifugation to produce a cell pellet and a culture supernatant, filtration, ultrafiltration, tangential flow-filtration, normal flow filtration or reverse osmosis. Alternatively, or in addition, the separation method can be ligand-based and include, for example, an antibody that specifically binds to Brevibacillus laterosporus. The antibody can be coupled to a solid support such as a magnetic bead.

It will be further apparent that the composition for use according to the present invention may comprise, for example, at least about 0.01%, about 0.05%, about 0.1%, about 0.2%, about 0.3%, about 0.4%, about 0.5%, about 0.6%, about 0.7%, about 0.8%, about 0.9%, about 1.0%, about 1.5%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 25.0%, about 30.0%, about 35.0%, about 40.0 about 45.0%, about 50.0% by weight of the fermentate thereof, and/or fraction thereof.

In one embodiment, the composition is a personal care composition or cosmetic composition, comprising a multifunctional fermentate, or fraction thereof, of Brevibacillus laterosporus.

In one embodiment, the composition is a personal care composition or cosmetic composition, comprising a multifunctional fermentate or fraction thereof, of a Brevibacillus laterosporus selected from the group consisting of Brevibacillus laterosporus G2 (CBS149108), Brevibacillus laterosporus A8.11 (CBS149109), Brevibacillus laterosporus ALS311 (CBS149785), Brevibacillus laterosporus ALS317 (CBS149786) Brevibacillus laterosporus ALS321 (CBS149788). In one embodiment, the composition is a personal care composition or cosmetic composition, comprising a multifunctional fermentate or fraction thereof, of Brevibacillus laterosporus strain A8.11.

In one aspect, the composition comprises a multifunctional fermentate of Brevibacillus laterosporus at about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or up to 10% by weight relative to a total weight of said composition

In one aspect, the composition comprises a multifunctional fermentate of Brevibacillus laterosporus G2 (CBS149108), Brevibacillus laterosporus A8.112 (CBS149109), Brevibacillus laterosporus ALS311 (CBS149785), Brevibacillus laterosporus ALS317, (CBS149786) or Brevibacillus laterosporus ALS321 (CBS149788) at about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or up to 10% by volume, relative to a total volume of said composition as well as a preservative at about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or up to 10% by weight relative to a total weight of said composition.

The preservative can be a traditional or a soft preservative.

In one aspect, the composition comprises an effective amount of a multifunctional fermentate of Brevibacillus laterosporus strain capable of enhancing the efficacy of a preservative.

In one embodiment the composition is a personal care composition comprising an effective amount of a Brevibacillus laterosporus multifunctional fermentate, or fraction thereof, and one or more preservative wherein the multifunctional fermentate enhances said preservative efficacy and provides one additional functionality selected from the group consisting of an antioxidant, a surfactant, a foam stabilizer, a foam enhancer, a UB blocker, a blue light blocker, and any one combination thereof.

The personal care composition of the present invention may further include one or more dermatologically or cosmetic acceptable component carrier.

In one embodiment, the composition is personal care or cosmetic composition comprising (a) an effective amount of a Brevibacillus laterosporus multifunctional fermentate, or fraction thereof, and (b) one or more preservative wherein each of (a) and (b) is present in the range of 0.001 to 5% by weight of the total composition, wherein said personal care or cosmetic composition further comprising one dermatologically or cosmetically active excipient.

In one embodiment, the composition is personal care or cosmetic composition comprising (a) an effective amount of a Brevibacillus laterosporus multifunctional fermentate, or fraction thereof, and (b) one or more preservative wherein each of (a) and (b) is present in the range of 0.001 to 5% by weight of the total composition, wherein said personal care or cosmetic composition further comprising one dermatologically or cosmetically active excipient, wherein said dermatologically or cosmetically active excipient is an anionic excipient and wherein said effective amount of a Brevibacillus laterosporus multifunctional fermentate, or fraction thereof, enhances said preservative efficacy

In one embodiment, the composition is personal care or cosmetic composition comprising (a) an effective amount of a Brevibacillus laterosporus multifunctional fermentate, or fraction thereof, and (b) one or more preservative wherein each of (a) and (b) is present in the range of 0.001 to 5% by weight of the total composition, wherein said personal care or cosmetic composition further comprising one dermatologically or cosmetically active excipient, wherein said dermatologically or cosmetically active excipient is an anionic excipient and wherein said effective amount of a Brevibacillus laterosporus multifunctional fermentate, or fraction thereof, enhances said preservative efficacy and further provide an additional function selected from the group consisting of an antioxidant, a surfactant, a foam stabilizer, a foam enhancer and any combination thereof.

In one aspect, the personal care composition described herein comprises an anionic excipient, wherein the anionic excipient, is present in the range of 0.001%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or up to 10% by weight relative to a total weight of said composition.

The personal care compositions of the present invention include, but are not limited to, cosmetic products (rinse-off cosmetic product, leave-on cosmetic products), aqueous solutions, emulsions, serums, jellies, patches, lotions, topical moisturizers, creams, pastes, balms, ointments, pomades, gels, liquids, sprays, foam, kits, or any one combinations thereof.

As used herein, the term “rinse-off product” refers to a cosmetic product which is intended to be removed after application on the skin, the hair, or mucous membranes. Rinse-off products include, but are not limited to, shower gels, shampoos, rinse-off conditioners, shower additives (salts, foams, oil, gel), shaving foam, shaving cream, shaving gel, or shaving soap.

As used herein, the term “leave-on product” refers to a cosmetic product which is intended to stay in prolonged contact with the skin, the hair, or the mucous membranes. Leave on products include, but are not limited to, make-up products, face creams, hair styling gels)

It will be further understood that the personal care composition for use in the present invention may further comprise one or more of probiotic bacteria in addition to the multifunctional fermentate, or fraction thereof, suitable for use in the present invention.

The personal care composition can comprise additional compounds selected from the group consisting of pH adjusters, antioxidants, surfactants, rheology modifiers, emollients, fragrances, and chelators.

pH adjusters include but are not limited to weak acids, strong acids, any compound that can adjust the pH, such as but not limiting to citric acid, or any combination thereof.

In one aspect, the personal care composition is formulated for topical administration to the skin/scalp.

The topical formulation (personal care composition) for use in the present invention may be in any form suitable for application to the subject in need, such as a cream, lotion, sprays, solution, gel, ointment, paste, plaster, paint, bioadhesive, suspensions or the like, and/or may be prepared so as to contain liposomes, micelles, and/or microspheres. Such a formulation may be used in combination with an occlusive overlayer so that moisture evaporating from the body surface is maintained within the formulation upon application to the body surface and thereafter.

Topical formulations include those in which the active ingredient(s) is (are) dissolved or dispersed in a dermatological vehicle known in the art (e.g. aqueous or non-aqueous gels, ointments, water-in-oil, or oil-in-water emulsions). Constituents of such vehicles may comprise water, aqueous buffer solutions, non-aqueous solvents (such as ethanol, isopropanol, benzyl alcohol, 2-(2-ethoxyethoxy) ethanol, propylene glycol, propylene glycol monolaurate, glycofurol or glycerol), oils (e.g. a mineral oil such as a liquid paraffin, natural or synthetic triglycerides, or silicone oils such as dimethicone). Depending, inter alia, upon the nature of the formulation as well as its intended use and site of application, the dermatological vehicle employed may contain one or more components (for example, when the formulation is an aqueous gel, components in addition to water) selected from the following list: a solubilizing agent or solvent (e.g. a β-cyclodextrin, such as hydroxypropyl β-cyclodextrin, or an alcohol or polyol such as ethanol, propylene glycol or glycerol); a thickening agent (e.g. hydroxyethylcellulose, hydroxypropylcellulose, carboxymethylcellulose or carbomer); a gelling agent (e.g. a polyoxyethylene-polyoxypropylene copolymer); a traditional or soft preservative; and pH buffering agent(s) (such as a mixture of dihydrogen phosphate and hydrogen phosphate salts, or a mixture of citric acid and a hydrogen phosphate salt).

The personal care composition of the present invention includes a liquid lotion (true solution) comprising water as a solvent and water soluble additives (solutes), such as but not limiting to an active, a fragrance, a color, a preservative, a pH adjuster, a chelating agent, or any one combination thereof.

The personal care composition of the present invention includes a dispersion such as an emulsion (such as, but not limited to the following: liquid in liquid [water in oil W/O, O/W, W/O/W], suspension [solid/liquid or liquid/solid], aerosol [liquid/gas or solid/gas], foam/mousse [gas/liquid or gas/emulsion, or gas/solid]). An example of an Oil in Water [O/W] emulsion includes but is not limited to a combination of a water phase, an emulsifier, a fatty phase and an at least one additive. The water phase can comprise water, humectants, and stabilizing agents [such as, but not limiting to, synthetic polymers, carbomers, natural polymers, xanthan gum, acacia gum, carragheenan, gellan, or any one combination thereof). Emulsifiers include, but are not limited to, anionic emulsifiers, cationic emulsifiers, non-ionic emulsifiers, amphoteric emulsifiers, silicone emulsifiers), auto emulsifying agents. Fatty phases (lipophilic ingredients) include, but are not limited to, waxes, butter, fatty esters, triglycerides, vegetal oil, mineral oil (parffinum), silicones, and thickeners/oil jellifying agents. Additives include, but are not limited to, preservative, fragrance (most often lipophilic), color, anti-oxidant, chelating agent, actives, pH adjuster (citric acid, lactic acid, AHA), neutralizers/strong basic agent like NaOH, Trimethylamine (for acrylic polymers to jellify) and powders.

The personal care composition of the present invention includes an aqueous gel comprising a water phase (including water, humectants, actives), a jellifying agent (such as but not limited to synthetic polymers, natural polymers, xanthan gum, acacia gum, carragheenan, gellan) and an additive (such as but not limited to fragrance, high HLB surfactant, color, actives, preservative system, pH adjuster, neutralizing agent, powders).

The personal care composition of the present invention includes a cleansing/surfactant system (such as but not limited to a shampoo, shower gel, micellar water) comprising a water phase (water, humectants), a surfactant, an additive (such as but not limited to fragrance, high HLB surfactant, color, actives, preservative system, pH adjuster, neutralizing agent, powders) and optionally a jellifying agent (such as but not limited to synthetic polymers, natural polymers, xanthan gum, acacia gum, carragheenan, gellan).

A dermatologically or cosmetically acceptable carrier may also be incorporated in the personal care composition of the present invention and may be any carrier conventionally used in the art. Examples thereof include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers.

The term “dermatologically acceptable” or “dermatologically acceptable carrier” or “dermatologically acceptable excipient” is used herein to refer to a compound or composition that may be incorporated into a dermatologically or personal care formulation without causing undesirable biological effects or unwanted interaction with other components of the formulation.

The term “cosmetically acceptable” or “cosmetically acceptable carrier” or “cosmetically acceptable excipient” is used herein to refer to a compound or composition that may be incorporated in a cometic formulation without causing undesirable biological effects or unwanted interaction with other components of the formulation.

“Carriers” or “vehicles” as used herein refer to carrier materials suitable for incorporation in a topically applied composition. Carriers and vehicles useful herein include any such materials known in the art, which are nontoxic and do not interact with other components of the formulation in which it is contained in a deleterious manner.

The term “aqueous” refers to a formulation that contains water or that becomes water-containing following application to the subject in need tissue (such as but not limiting to skin or mucosal tissue).

Personal care composition of the present invention may further comprise one or more dermatologically acceptable components known or otherwise effective for use in personal care products, provided that the optional components are physically and chemically compatible with the essential components described herein, or do not otherwise unduly impair product stability, aesthetics, or performance. Non-limiting examples of such optional components are disclosed in International Skin Care Ingredient Dictionary, Ninth Edition, 2002, and CTFA Skin Care Ingredient Handbook, Tenth Edition, 2004.

In one aspect, the dermatologically acceptable component is a dermatologically acceptable component comprising from about 10 wt. % to about 99.9 wt. %, alternatively from about 50 wt. % to about 95 wt. %, and alternatively from about 75 wt. % to about 95 wt. %, of a dermatologically acceptable carrier. Carriers suitable for use with the composition(s) may include, for example, those used in the formulation of mousses, tonics, gels, skin moisturizers and lotions. The carrier may comprise water; organic oils; silicones such as volatile silicones, amino or non-amino silicone gums or oils, and mixtures thereof; mineral oils; plant oils such as olive oil, castor oil, rapeseed oil, coconut oil, wheat germ oil, sweet almond oil, avocado oil, macadamia oil, apricot oil, safflower oil, candlenut oil, false flax oil, tamanu oil, lemon oil and mixtures thereof; waxes; and organic compounds such as C₂-C₁₀ alkanes, acetone, methyl ethyl ketone, volatile organic C₁-C₁₂ alcohols, esters of C₁-C₂₀ acids and of C₁-C₈ alcohols such as methyl acetate, butyl acetate, ethyl acetate, and isopropyl myristate, dimethoxyethane, diethoxyethane, C₁₀-C₃₀ fatty alcohols such as lauryl alcohol, cetyl alcohol, stearyl alcohol, and behenyl alcohol; C₁₀-C₃₀ fatty acids such as lauric acid and stearic acid; C₁₀-C₃₀ fatty amides such as lauric diethanolamide; C₁₀-C₃₀ fatty alkyl esters such as C₁₀-C₃₀ fatty alkyl benzoates; hydroxypropylcellulose, and mixtures thereof. In one aspect, the carrier comprises water, fatty alcohols, volatile organic alcohols, and mixtures thereof. Other carriers can be formulated by those of ordinary skill in the art.

The personal care composition of the present invention described herein may further comprise from about 0.1% to about 10%, and alternatively from about 0.2% to about 5.0%, of a gelling agent to help provide the desired viscosity to the composition(s). Non-limiting examples of suitable optional gelling agents include alginates, xanthan gum, crosslinked carboxylic acid polymers; unneutralized crosslinked carboxylic acid polymers; unneutralized modified crosslinked carboxylic acid polymers; crosslinked ethylene/maleic anhydride copolymers; unneutralized crosslinked ethylene/maleic anhydride copolymers (e.g., EMA 81 commercially available from Monsanto); unneutralized crosslinked alkyl ether/acrylate copolymers (e.g., SALCARE™ SC90 commercially available from Allied Colloids); unneutralized crosslinked copolymers of sodium polyacrylate, mineral oil, and PEG-1 trideceth-6 (e.g., SALCARE™ SC91 commercially available from Allied Colloids); unneutralized crosslinked copolymers of methyl vinyl ether and maleic anhydride (e.g., STABILEZE™ QM-PVM/MA copolymer commercially available from International Specialty Products); hydrophobically modified nonionic cellulose polymers; hydrophobically modified ethoxylate urethane polymers (e.g., UCARE™ Polyphobe Series of alkali swellable polymers commercially available from Union Carbide); and combinations thereof. In this context, the term “unneutralized” means that the optional polymer and copolymer gelling agent materials contain unneutralized acid monomers.

The dermatologically or cosmetically acceptable medium may contain a fatty substance in a proportion generally of from about 10 to about 90% by weight relative to the total weight of the product, where the fatty phase containing at least one liquid, solid or semi-solid fatty substance. The fatty substance includes, but is not limited to, oils, waxes, gums, and so-called pasty fatty substances. Alternatively, the products may be in the form of a stable dispersion such as a water-in-oil or oil-in-water emulsion. Additionally, the personal care products may contain one or more conventional cosmetic or dermatological additives or adjuvants, including but not limited to, antioxidants, preserving agents, fillers, surfactants, UVA and/or UVB sunscreens, fragrances, thickeners, wetting agents and anionic, nonionic or amphoteric polymers, and dyes or pigments (colorant agents).

The dermatologically acceptable carrier may be a moisturizer formulation containing at least one emulsifier, at least one surfactant, or any combination thereof.

Personal care compositions described herein can further comprise active ingredient materials including sun screen agents, moisturizers, humectants, benefiting agents skin, depositing agents such as surfactants, occlusive agents, moisture barriers, lubricants, emollients, anti-aging agents, antistatic agents, abrasive, antimicrobials, conditioners, exfoliants, fragrances, viscosifying agents, salts, lipids, phospholipids, vitamins, foam stabilizers, pH modifiers, preservatives, suspending agents, silicone oils, silicone derivatives, essential oils, oils, fats, fatty acids, fatty acid esters, fatty alcohols, waxes, polyols, hydrocarbons, and mixtures thereof.

In one embodiment, the composition is a personal care composition comprising an effective amount of a Brevibacillus laterosporus multifunctional fermentate, or fraction thereof, and, a preservative, wherein the personal care composition is a synergistic personal care composition comprising a combination of an effective amount of a Brevibacillus laterosporus multifunctional fermentate, or fraction thereof and, a preservative wherein said combination provides a synergistic preservation effect in said composition.

In one embodiment, the synergistic personal care composition is a cream comprising a synergistic combination of at least 0.3% preservative and at least 2.5% of a Brevibacillus laterosporus multifunctional fermentate.

In one embodiment, the synergistic personal care composition is a cream comprising a synergistic combination of at least 0.3% preservative and at least 5.0% of a Brevibacillus laterosporus multifunctional fermentate.

In one embodiment, the synergistic personal care composition is a lotion comprising a synergistic combination of at least 0.4% preservative and at least 1.0% of a Brevibacillus laterosporus multifunctional fermentate.

In one embodiment, the synergistic personal care composition is a lotion comprising a synergistic combination of at least 0.4% preservative and at least 2.5% of a Brevibacillus laterosporus multifunctional fermentate.

In one embodiment, the synergistic personal care composition is a serum comprising a synergistic combination of at least 0.4% preservative and at least 2.5% of a Brevibacillus laterosporus multifunctional fermentate.

In one embodiment, the synergistic personal care composition is a serum comprising a synergistic combination of at least 0.5% preservative and at least 2.5% of a Brevibacillus laterosporus multifunctional fermentate.

In one embodiment, the personal care composition is a skin care composition formulated for administration to the skin.

In one embodiment the composition is a skin care composition comprising an effective amount of a Brevibacillus laterosporus multifunctional fermentate, or fraction thereof, and one or more preservative wherein the multifunctional fermentate enhances said preservative efficacy and provides one additional functionality selected from the group consisting of an antioxidant, a surfactant, a foam stabilizer, a foam enhancer, a UB blocker, a blue light blocker, and any one combination thereof.

As used herein the term “skin care composition(s)” refers to composition(s) comprising at least one skin care benefit agent (active agent) capable of providing a skin care benefit.

As used herein the term “skin care benefit agent” or “active agent” are used interchangeably and refer to a compound that can provide a skin care benefit.

As used herein the term “skin care benefit” refers to a benefit provided by an active agent (or skin care composition comprising an effective amount of said active agent) when applied topically to a skin.

Other ingredients that may be included in a skin care composition or skin care product include, without limitation, at least one active ingredient for the treatment or prevention of skin ailments, providing a skin care effect, or for providing a moisturizing benefit to skin, such as zinc oxide, petrolatum, white petrolatum, mineral oil, cod liver oil, lanolin, dimethicone, hard fat, vitamin A, allantoin, calamine, kaolin, glycerin, or colloidal oatmeal, and combinations of these, one or more natural moisturizing factors (such as betaine, xylitol, panthenol, ceramides, hyaluronic acid, glycerin, squalane, amino acids, cholesterol, fatty acids, triglycerides, phospholipids, glycosphingolipids, urea, linoleic acid, glycosaminoglycans, mucopolysaccharide, sodium lactate, or sodium pyrrolidone carboxylate, for example), glycerides, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, cholesterol, cholesterol esters, wax esters, fatty acids, and orange oil.

Any number of dermatologically acceptable materials commonly used in skin care products may also be incorporated into the present skin care products such as skin conditioning agents and skin colorants.

Skin conditioning agents as herein defined include, but are not limited to astringents, which tighten skin; exfoliants, which remove dead skin cells; emollients, which help maintain a smooth, soft, pliable appearance; humectants, which increase the water content of the top layer of skin; occlusives, which retard evaporation of water from the skin's surface; and miscellaneous compounds that enhance the appearance of dry or damaged skin or reduce flaking and restore suppleness. Skin conditioning agents are well known in the art, see for example Green et al. (WO01/07009), and are available commercially from various sources. Suitable examples of skin conditioning agents include, but are not limited to, lactobionic acid, gluconic acid, alpha-hydroxy acids, beta-hydroxy acids, polyols, hyaluronic acid, D,L-panthenol, polysalicylates, vitamin A palmitate, vitamin E acetate, glycerin, sorbitol, silicones, silicone derivatives, lanolin, natural oils, xylitol, fucose, rhamnose, and triglyceride esters. The skin conditioning agents may include polysalicylates, propylene glycol (CAS No. 57-55-6, Dow Chemical, Midland, MI), glycerin (CAS No. 56-81-5, Proctor & Gamble Co., Cincinnati, OH), glycolic acid (CAS No. 79-14-1, DuPont Co., Wilmington, DE), lactic acid (CAS No. 50-21-5, Alfa Aesar, Ward Hill, MA), malic acid (CAS No. 617-48-1, Alfa Aesar), citric acid (CAS No. 77-92-9, Alfa Aesar), tartaric acid (CAS NO. 133-37-9, Alfa Aesar), glucaric acid (CAS No. 87-73-0), galactaric acid (CAS No. 526-99-8), 3-hydroxyvaleric acid (CAS No. 10237-77-1), salicylic acid (CAS No. 69-72-7, Alfa Aesar), and 1,3 propanediol (CAS No. 504-63-2, DuPont Co., Wilmington, DE). Polysalicylates may be prepared by the method described by White et al. in U.S. Pat. No. 4,855,483, incorporated herein by reference. Glucaric acid may be synthesized using the method described by Merbouh et al. (Carbohydr. Res. 336:75-78 (2001). The 3-hydroxyvaleric acid may be prepared as described by Bramucci in published international patent application number WO 02/012530.

Skin care compositions can comprise skin care additives such as, but not limiting to, colorants/dyes, fragrances, actives, preservatives, pH adjusters, chelators, and antioxidants.

Personal care compositions described herein can also be part of a kit for providing one or more personal care benefits such as, but not limiting to, a kit for preventing or reducing a dandruff condition.

In one aspect the kit is a kit comprising a multifunctional fermentate of Brevibacillus laterosporus, or fraction thereof, wherein said composition enhances the efficacy of a preservative and simulataneously provide at least one additional function selected from the group consisting of an antioxidant, a surfactant, a foam stabilizer, a foam enhancer, a UB blocker, a blue light blocker, and any one combination thereof.

Methods for Providing Multiple Functions to a Composition while Enhancing the Efficacy of a Preservative

The personal care compositions described herein can be used in methods for providing multiple functional properties to a composition, the method comprising adding an effective amount of a Brevibacillus laterosporus multifunctional fermentate, or fraction thereof, and one or more preservative to a composition, wherein the multifunctional fermentate enhances said preservative efficacy while simultaneously providing an additional function selected form the group consisting of an antioxidant agent, an emulsifier, a product stabilizer, a rheology modifier, emulsification, a thickener, or any one combination thereof.

In one embodiment, the method is a method for enhancing the efficacy of a preservative in a composition, the method comprising adding an effective amount of a Brevibacillus laterosporus multifunctional fermentate, or fraction thereof, to a composition comprising a preservative, wherein each of said preservative and said fermentate are present in the range of 0.001% to 5.0% by weight of the total composition, wherein the multifunctional fermentate enhances said preservative efficacy.

In one embodiment, the method is a method for enhancing the efficacy of a preservative in a composition while simultaneously providing an additional function to said composition, the method comprising adding an effective amount of a Brevibacillus laterosporus multifunctional fermentate, or fraction thereof, to a composition comprising a preservative, wherein each of said preservative and said fermentate are present in the range of 0.001% to 5.0% by weight of the total composition, wherein the multifunctional fermentate enhances said preservative efficacy while simultaneously providing an additional function selected form the group consisting of an antioxidant agent, a surfactant, a foam stabilizer, a foam enhancer, a UV blocker, a blue light blocker, or any one combination thereof.

In one embodiment, the method is a method for enhancing the efficacy of a preservative in a composition, the method comprising adding a combination of an effective amount of a Brevibacillus laterosporus multifunctional fermentate, or fraction thereof and, a preservative wherein said combination provides a synergistic preservation effect in said composition.

### General Definitions

The disclosures of all cited patent and non-patent literature are incorporated herein by reference in their entirety.

In this disclosure, a number of terms and abbreviations are used. The following definitions apply unless specifically stated otherwise.

As used herein, the articles “a”, “an”, and “the” preceding an element or component of the invention are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

When an amount, concentration, or other value or parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope be limited to the specific values recited when defining a range.

The use of numerical values in the various ranges specified in this application, unless expressly indicated otherwise, are stated as approximations as though the minimum and maximum values within the stated ranges were both proceeded by the word “about”. In this manner, slight variations above and below the stated ranges can be used to achieve substantially the same results as values within the ranges. Also, the disclosure of these ranges is intended as a continuous range including each and every value between the minimum and maximum values. As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

As used herein “administer” or “administering” is meant the action of introducing one or more microorganism (microbial strain), one or more fermentate, or fraction thereof of a microorganism, personal care composition(s), personal care formulation(s) and/or personal care product(s) to a subject.

As used herein, the term “biological contaminants” refers to one or more unwanted and/or pathogenic biological entities including, but not limited to, microorganisms, spores, viruses, prions, and mixtures thereof.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “embodiment” or “disclosure” is not meant to be limiting but applies generally to any of the embodiments defined in the claims or described herein. These terms are used interchangeably herein.

As used herein, the term “cell lysate” or “lysate” refers to microbial cells which have been lysed by any suitable means. The term “cell lysate” or “lysate” conventionally denotes a material obtained after the destruction or dissolution of biological cells via a phenomenon known as cell lysis, thus giving rise to the release of the intracellular biological constituents naturally contained in the cells of the microorganism under consideration. For the purposes of the present disclosure, the term “lysate” is used without preference to denote the whole lysate obtained via lysis of the microorganism under consideration or only a fraction thereof. The lysate used is thus totally or partially formed from the intracellular biological constituents and from the constituents of the cell walls and membranes. A lysate used for the invention may be the whole lysate obtained via lysis of the microorganism under consideration, or a fraction thereof. This cell lysis may be accomplished by any suitable means or any one method known in the art, such as but not limiting to, an osmotic shock, a heat shock, ultrasonication, sonication, homogenization, shearing, chemical lysis or under a mechanical stress of centrifugation type. In some embodiments, the cell debris is removed from the cell lysate prior to use. In some embodiments the cell lysates are filtered or fractionated prior to use.

As used herein, the term “excipient” refers to inactive substance used as a carrier for active ingredients, in a formulation. The excipient may be used to stabilize the active ingredient in a formulation, such as the storage stability of the active ingredient. Excipients are also sometimes used to bulk up formulations that contain active ingredients.

As used herein, the term “effective amount” refers to the amount sufficient to obtain the desired effect.

As used herein, the term “metabolite(s) thereof” or “metabolite(s) of the microorganism(s) suitable for use in the present invention” or “metabolite actives” are used interchangeably and refer to any substance derived from the metabolism of a microorganism(s) suitable for use in the present invention

As used herein, the term “soluble metabolite” refers to a metabolite or metabolites present in the supernatant of a cell culture (fermentate supernatant) from which the cells have been removed. In one embodiment the cells are removed by centrifugation. In one embodiment the supernatant is filtered. It will be apparent that the supernatant may be used directly in the formulations of the present invention, or that one or more of the metabolites may be isolated form the supernatant by any suitable means prior to use.

As used herein, the term “reducing”, “reduces” and grammatical variations thereof in relation to a particular trait, characteristic, feature, biological process, or phenomena refers to a decrease in the particular trait, characteristic, feature, biological process, or phenomena. The trait, characteristic, feature, biological process, or phenomena can be decreased by 5%, 10%, 15%, 20%, 25%, 30%, 35%, 40%, 45%, 50%, 55%, 60%, 65%, 70%, 75%, 80%, 85%, 90%, 95%, 100% or greater than 100%.

The terms “percent by weight”, “weight percentage (wt. %)” and “weight-weight percentage (% w/w)” are used interchangeably herein. Percent by weight refers to the percentage of a material on a mass basis as it is comprised in a composition, mixture, solution, or product.

The terms “percent by volume”, “volume percentage” are used interchangeably herein. Percent by volume refers to the percentage of a material on a volume basis as it is comprised in a composition, mixture, solution, or product.

The term “16S rRNA” or “16S ribosomal RNA” means the rRNA constituting the small subunit of prokaryotic ribosomes. In bacteria, this sequence can be used to identify and characterize operational taxonomic units.

The term “ITS” or “Internal Transcribed Spacers” are regions within the ribosomal transcript that are excised and degraded during maturation. Their sequences can be used for phylogenetic analysis and/or identification of fungi or yeast.

As used herein the term “synergistic personal care composition” refers to a composition comprising a combination of an effective amount of a Brevibacillus laterosporus multifunctional fermentate, or fraction thereof; and, a preservative wherein said combination provides a synergistic preservation effect in said composition.

In one aspect, a “synergistic personal care composition” refers to a composition comprising of an effective amount of a Brevibacillus laterosporus multifunctional fermentate, or fraction thereof; and, a preservative wherein the combined preservative effect is greater than the sum of the individual preservative effects of each of said Brevibacillus laterosporus multifunctional fermentate and preservative (thus providing a synergistic effect).

In one aspect, a “synergistic skin care composition” refers to a composition comprising an effective amount of at least two different human milk oligosaccharides (at a total HMO concentration of X), that provides a skin care benefit that is greater than the skin care benefit achieved by using either individual HMO alone at said same total HMO concentration of X.

The terms moisturizer, a lotion or a body lotion refer to a low to medium-viscosity emulsion of oil and water, most often oil-in-water but possibly water-in-oil with the primary benefit in a personal care application to hydrate the subject in need or to reduce its water loss. Nearly all moisturizers contain a combination of emollients, occlusives, and humectants. Emollients, which are mainly lipids and oils, hydrate and improve the appearance of the skin. A wide variety of suitable emollients is known and maybe used herein (International Skin Care Ingredient Dictionary and Handbook, eds. Wenninger and McEwen, pp. 1656-61, 1626, and 1654-55 (The Skin care, Toiletry, and Fragrance Assoc., Washington, D.C., 7th Edition, 1997) (referred to as “ICI Handbook”) contains numerous examples of suitable materials). Occlusives such as petrolatum, lanolin and bee wax reduce transepidermal water loss by creating hydrophobic barrier over the skin. Humectants such as glycerol and urea able to attract water from the external environment and enhance water absorption from the dermis into the epidermis. In addition, the moisturizer formulations may contain emulsifiers to maintain stability of emulsions and use thickeners to achieve desired viscosity and skin feel. A wide variety of other ingredients such as fragrances, dyes, preservatives, therapeutic agents, proteins, and stabilizing agents are commonly added for other consumer preferred attributes.

The term “percent (%) sequence identity” or “percent (%) sequence similarity,” as used herein with respect to a reference sequence is defined as the percentage of nucleotide residues in a candidate sequence that are identical to the residues in the reference polynucleotide sequence after optimal alignment of the sequences and introducing gaps, if necessary, to achieve the maximum percent sequence identity.

A microbial “strain” as used herein refers to a microorganism (such as a bacterium or fungus) which remains genetically unchanged when grown or multiplied. The multiplicity of identical microbes is included.

As used herein, the term a “biologically pure strain” means a strain containing no other microbial strains in quantities sufficient to interfere with replication of the strain or to be detectable by normal techniques. “Isolated” when used in connection with the organisms and cultures described herein includes not only a biologically pure strain, but also any culture of organisms which is grown or maintained other than as it is found in nature.

As used herein, the term “probiotic” or “probiotic microorganism” are used interchangeably herein and refer to a live microorganism (including bacteria or yeasts for example) which, when administered (topically or orally) in sufficient amounts, beneficially affects the host organism, i.e. by conferring one or more demonstrable benefits, such as a reduced dandruff condition, on the host organism. Whilst there are no lower or upper limits for probiotic use, it has been suggested that at least 10⁶-10¹², preferably at least 10⁶-10¹⁰, preferably 10⁸-10⁹, cfu as a daily dose will be effective to achieve the beneficial effects in a subject.

The term “sequence identity” or “sequence similarity” as used herein, means that two polynucleotide sequences, a candidate sequence and a reference sequence, are identical (i.e. 100% sequence identity) or similar (i.e. on a nucleotide-by-nucleotide basis) over the length of the candidate sequence. In comparing a candidate sequence to a reference sequence, the candidate sequence may comprise additions or deletions (i.e. gaps) as compared to the reference sequence (which does not comprise additions or deletions) for optimal alignment of the two sequences. Optimal alignment of sequences for determining sequence identity may be conducted using the any number of publicly available local alignment algorithms known in the art such as ALIGN or Megalign (DNASTAR), or by inspection.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Non-limiting examples of compositions and methods disclosed herein include:
1) A composition comprising: a) an effective amount of a Brevibacillus laterosporus multifunctional fermentate, or fraction thereof; and, b) one or more preservative, wherein each of (a) and (b) is present in the range of 0.001% to 5% by weight of the total composition.
2) The composition of embodiment 1, wherein the multifunctional fermentate or fraction thereof, enhances said preservative efficacy.
2b) The composition of embodiment 1, wherein the multifunctional fermentate enhances said preservative efficacy by reducing the preservative's use level needed to be effective.
2c) The composition of embodiment 1, wherein the multifunctional fermentate enhances said preservative efficacy by reducing the preservative's use (dosing) level needed to be effective compared to the preservative use level needed if said multifunctional was absent of said composition.
2d) The composition of embodiment 1, wherein said preservative is a soft preservative.
3) The composition of embodiment 1 or embodiment 2, wherein the multifunctional fermentate, or fraction thereof, has at least two functions selected from the group consisting of an antioxidant, a surfactant, a foam stabilizer, a foam enhancer, a UB blocker, a blue light blocker, an enhancer of one or more preservatives, and any one combination thereof.
   3b) The composition of embodiment 1 or embodiment 2, wherein Brevibacillus laterosporus is selected from the group consisting of a Brevibacillus laterosporus having a 16S ribosomal RNA sequence displaying at least 97.0% sequence similarity to a 16S ribosomal RNA sequence of Brevibacillus laterosporus strain G2 (SEQ ID NO: 1) deposited at Westerdijk Fungal Biodiversity Institute (WFDB) under number CBS149108; a Brevibacillus laterosporus having a 16S ribosomal RNA sequence displaying at least 97.0% sequence similarity to a 16S ribosomal RNA sequence of non-sporulating Brevibacillus laterosporus strain A8.11 derived from Brevibacillus laterosporus (SEQ ID NO: 1) deposited at Westerdijk Fungal Biodiversity Institute (WFDB) under number CBS149109; a Brevibacillus laterosporus suitable for use in the present invention includes, but is not limited to, a Brevibacillus laterosporus having a 16S ribosomal RNA sequence displaying at least 97.0% sequence similarity to a 16S ribosomal RNA sequence of Brevibacillus laterosporus ALS311 (SEQ ID NO: 1) deposited at Westerdijk Fungal Biodiversity Institute (WFDB) under number CBS149785; a Brevibacillus laterosporus having a 16S ribosomal RNA sequence displaying at least 97.0% sequence similarity to a 16S ribosomal RNA sequence of Brevibacillus laterosporus ALS317 (SEQ ID NO: 1) deposited at Westerdijk Fungal Biodiversity Institute (WFDB) under number CBS149786; and a Brevibacillus laterosporus having a 16S ribosomal RNA sequence displaying at least 97.0% sequence similarity to a 16S ribosomal RNA sequence of Brevibacillus laterosporus ALS321 (SEQ ID NO: 1) deposited at Westerdijk Fungal Biodiversity Institute (WFDB) under number CBS149788.
4) The composition of any preceding embodiments, wherein the composition is a personal care or cosmetic composition further comprising one dermatologically or cosmetically active excipient.
4b) A personal care or cosmetic composition comprising the composition of embodiment 1 and one dermatologically or cosmetically active excipient.
5) The personal care composition of embodiment 4, wherein said dermatologically or cosmetically active excipient is an anionic excipient and wherein said effective amount of a Brevibacillus laterosporus multifunctional fermentate, or fraction thereof, enhances said preservative efficacy.
5b) A personal care composition comprising an anionic excipient and an effective amount of a Brevibacillus laterosporus multifunctional fermentate, or fraction thereof, wherein the multifunctional fermentate or fraction thereof, enhances said preservative efficacy.
6) The personal care composition of embodiment 4, wherein said dermatologically or cosmetically active excipient is an anionic excipient and wherein said effective amount of a Brevibacillus laterosporus multifunctional fermentate, or fraction thereof, enhances said preservative efficacy and further provide an additional function selected from the group consisting of an antioxidant, a surfactant, a foam stabilizer, a foam enhancer and any combination thereof.
6b) The personal care composition of embodiment 6, wherein the multifunctional fermentate or fraction thereof, enhances said preservative efficacy and has at least one additional function selected from the group consisting of an antioxidant, a surfactant, a foam stabilizer, a foam enhancer, a UB blocker, a blue light blocker, an enhancer of one or more preservatives, and any one combination thereof.
6b) A personal care composition comprising an anionic excipient and an effective amount of a Brevibacillus laterosporus multifunctional fermentate, or fraction thereof, wherein the multifunctional fermentate has at least two functions selected from the group consisting of an antioxidant, a surfactant, a foam stabilizer, a foam enhancer, a UB blocker, a blue light blocker, an enhancer of one or more preservatives, and any one combination thereof.
7) The personal care composition of embodiment 5, wherein the anionic excipient, is present in the range of 0.001% to 5% by weight of the total composition
8) The personal care composition of (embodiments 4-7) embodiment 4, wherein said composition is in the form of a serum, a hydrogel, a cream, an emulsion, a lotion, a gel, an oil, a wipe, an ointment, a semi-solid formulation, or an aerosol spray.
8b) The personal care composition of embodiment 4, wherein the composition is a rinse-off cosmetic product.
8c) The personal care composition of embodiment 4, wherein the composition is a leave-on cosmetic product.
9) The personal care composition of embodiment 4, wherein the composition is topically applied.
10) The composition of embodiment 4, wherein the composition is a synergistic personal care composition comprising a combination of an effective amount of a Brevibacillus laterosporus multifunctional fermentate, or fraction thereof; and, a preservative, and wherein said combination provides a synergistic preservation effect of said composition.
11) The composition of embodiment 1, wherein the preservative is selected from the group consisting of phenoxyethanol, benzoic acid, sorbic acid, citric acid, sodium benzoate, potassium sorbate, phenyl ethyl alcohol, lauryl ethyl arginate and any combination thereof.
12) A method for enhancing the efficacy of a preservative in a composition, the method comprising adding an effective amount of a Brevibacillus laterosporus multifunctional fermentate, or fraction thereof, to a composition comprising a preservative, wherein each of said preservative and said fermentate are present in the range of 0.001% to 5.0% by weight of the total composition, wherein the multifunctional fermentate enhances said preservative efficacy.
13) A method for enhancing the efficacy of a preservative in a composition while simultaneously providing an additional function to said composition, the method comprising adding an effective amount of a Brevibacillus laterosporus multifunctional fermentate, or fraction thereof, to a composition comprising a preservative, wherein each of said preservative and said fermentate are present in the range of 0.001% to 5.0% by weight of the total composition, wherein the multifunctional fermentate enhances said preservative efficacy while simultaneously providing an additional function selected form the group consisting of an antioxidant agent, a surfactant, a foam stabilizer, a foam enhancer. A UB blocker, a blue light blocker, or any one combination thereof.
14) The method of embodiment 12, wherein the preservative is selected from the group consisting of phenoxyethanol, benzoic acid, sorbic acid, citric acid, sodium benzoate, potassium sorbate, phenyl ethyl alcohol, lauryl ethyl arginate and any combination thereof.
15) The method of embodiment 12, wherein the composition is a personal care or cosmetic composition.
16) The method of embodiment 15, wherein said personal care composition is in the form of a serum, a hydrogel, a cream, an emulsion, a lotion, a gel, an oil, a wipe, an ointment, a semi-solid formulation, or an aerosol spray.
17) The method of embodiment 15, wherein the composition is topically applied.
18) Use of an effective amount of a Brevibacillus laterosporus multifunctional fermentate in a personal care formulation, wherein said multifunctional fermentate has at least two functions selected from the group consisting of an antioxidant agent, a surfactant, a foam stabilizer, a foam enhancer, a UB blocker, a blue light blocker, an enhancer of one or more preservatives, or any one combination thereof.
19) The composition of embodiment 1, wherein the Brevibacillus laterosporus multifunctional fermentate is selected from the group consisting of a liquid fermentate, a spray dried fermentate or an encapsulated fermentate.
20) The method of embodiment 1, wherein the Brevibacillus laterosporus multifunctional fermentate is selected from the group consisting of a liquid fermentate, a spray dried fermentate or an encapsulated fermentate.
21. The personal care composition of embodiment 4, wherein said personal care composition has less than 500 ppb of a preservative listed in Annex VI. (see WO201704534A2)
22. The personal care composition of embodiment 4, wherein said personal care composition does not include a preservative listed in Annex VI. (see WO201704534A2) 23. A Brevibacillus laterosporus multifunctional fermentate, wherein said multifunctional fermentate has at least two activities selected from the group consisting of an antioxidant agent, a surfactant, a foam stabilizer, a foam enhancer, a UB blocker, a blue light blocker, an enhancer of one or more soft preservatives, and any one combination thereof.
24) A Brevibacillus laterosporus multifunctional fermentate, wherein said multifunctional fermentate enhances the efficacy of a soft preservative.
25) A synergistic personal care composition comprising a combination of an effective amount of a Brevibacillus laterosporus multifunctional fermentate, or fraction thereof and, a preservative wherein said combination provides a synergistic preservation effect in said composition.
26) The synergistic personal care composition of embodiment 25, wherein the composition is a cream comprising a synergistic combination of at least 0.3% preservative and at least 2.5% of a Brevibacillus laterosporus multifunctional fermentate.
27) The synergistic personal care composition of embodiment 25, wherein the composition is a cream comprising a synergistic combination of at least 0.3% preservative and at least 5.0% of a Brevibacillus laterosporus multifunctional fermentate.
28) The synergistic personal care composition of embodiment 25, wherein the composition is a lotion comprising a synergistic combination of at least 0.4% preservative and at least 1.0% of a Brevibacillus laterosporus multifunctional fermentate.
29) The synergistic personal care composition of embodiment 25, wherein the composition is a lotion comprising a synergistic combination of at least 0.4% preservative and at least 2.5% of a Brevibacillus laterosporus multifunctional fermentate.
30) The synergistic personal care composition of embodiment 25, wherein the composition is a serum comprising a synergistic combination of at least 0.4% preservative and at least 2.5% of a Brevibacillus laterosporus multifunctional fermentate.
31) The synergistic personal care composition of embodiment 25, wherein the composition is a serum comprising a synergistic combination of at least 0.5% preservative and at least 2.5% of a Brevibacillus laterosporus multifunctional fermentate.

### EXAMPLES

In the following Examples, unless otherwise stated, parts and percentages are by weight and degrees are Celsius. It should be understood that these Examples, while indicating embodiments of the disclosure, are given by way of illustration only. From the above discussion and these Examples, one skilled in the art can make various changes and modifications of the disclosure to adapt it to various usages and conditions. Such modifications are also intended to fall within the scope of the appended claims.

The following abbreviations in the specification correspond to units of measure, techniques, properties, or compounds as follows: “sec” or “s” means second(s), “min” means minute(s), “h” or “hr” means hour(s), “μL” means microliter(s), “mL” means milliliter(s), “L” means liter(s), “mM” means millimolar, “M” means molar, “mmol” means millimole(s), “ppm” means part(s) per million, “wt” means weight, “wt %” means weight percent, “g” means gram(s), “mg” means milligram(s), “μg” means microgram(s), “ng” means nanogram(s), “conc.” means concentration, “Trt” means treatment.

### Example 1

### Brevibacillus laterosporus Strains

Brevibacillus laterosporus suitable for use in the present invention includes Brevibacillus laterosporus having a 16S ribosomal RNA sequence displaying at least 97.0% sequence similarity to a 16S ribosomal RNA sequence of Brevibacillus laterosporus G2 (SEQ ID NO: 1) deposited at Westerdijk Fungal Biodiversity Institute (WFDB) under number CBS 149108. Brevibacillus laterosporus strain G2 was isolated from a facial skin microbiome library prepared by swabbing the surface of facial skin, transferring to phosphate-buffered saline and serially diluted. Serial dilutions were plated onto petri dishes containing TSA (17 g Tryptone, 3 g Soytone, 5 g NaCl, 2.5 g dipotassium phosphate, 2.5 g glucose, 15 g agar per liter distilled water) media. Plates were then incubated aerobically at 33° C. until colonies became visible. Isolated colonies were the source for PCR reactions using standard protocols. The phylogenetic identity of Brevibacillus laterosporus strain G2 was determined by sequencing the 16S region with primer set A (SEQ ID NO: 2) where “M” is nucleotide Adenine or Cytosine and primer set B (SEQ ID NO: 3).

### Isolation of an Endospore Deficient Strain

Brevibacillus laterosporus is a rod-shaped, endospore-forming bacterium. It is not, however, desirable for endospores to be present in a final product or waste from industrial production. Therefore, it is preferred that the Brevibacillus laterosporus production strain is incapable of forming endospores.

Eliminating endospore production was accomplished using a homologous recombination vector targeting the Stage II, sporulation protein E (spoIIE) gene of Brevibacillus laterosporus (SEQ ID NO: 4). The spoIIE locus of Bacillus subtilis is required for formation of a normal endospore through activation of the transcription factor sigma F (Barák I., Behari J., Olmedo G., Guzmán P., Brown D., Castro E., Walker D., Westpheling J., & Youngman P. (1996) Structure and function of the Bacillus SpoIIE protein and its localization to sites of sporulation septum assembly. Mol Microbiol. 19 (5): 1047-60.). A truncated spoIIE gene was synthesized, by eliminating the first 600 bp, beginning with the initiation codon (SEQ ID NO: 5; Integrated DNA Technologies, Coralville, Iowa). The truncated gene was assembled into an integration vector (Leenhouts, K. J., et. al, 1991, Plasmid, 26 (1), 55-66; Maguin, E., et al., 1992. J Bacteriol, 174 (17), 5633-5638)

Successful integration, resulting in truncation of the spoIIE gene, was determined by PCR using the primer set primer set A2 (SEQ ID NO: 6) and primer set B2 (SEQ ID NO: 7). The deleted spoIIE sequence was confirmed by DNA sequencing. The absence of endospore production in the knockout strain was determined visually following growth in spore induction media and compared to the parent Brevibacillus laterosporus G2 strain (overnight cultures in TSB media were harvested by centrifugation and inoculated into sporulation medium (8 g/L Nutrient broth, 1 g/L KCl, 1 mM MgSO4, 1 mM Ca(NO3)2, 10 μM MnCl2, 1 μM FeSO4). Cells were grown at 33° C. with agitation (200 rpm) and observed for production of spores after 48 hours).

The spoIIE deletions strain, demonstrated to be endospore deficient, was designated Brevibacillus laterosporus A8.11 and deposited at Westerdijk Fungal Biodiversity Institute (WFDB) under number CBS149109.

Brevibacillus laterosporus suitable for use in the present invention includes Brevibacillus laterosporus having a 16S ribosomal RNA sequence displaying at least 97.0% sequence similarity to a 16S ribosomal RNA sequence of Brevibacillus laterosporus A8.11 (SEQ ID NO: 1) deposited at Westerdijk Fungal Biodiversity Institute (WFDB) under number CBS149109.

Knockout of Non-Essential Genes in Brevibacillus laterosporus

Brevibacillus laterosporus strains contains genes associated with antibiotic resistance. In one aspect, elimination of genes that may confer resistance to antibiotics is desired, especially for Brevibacillus laterosporus production strains (strains used for large scale/commercial production of Brevibacillus laterosporus fermentates). Eliminating a series of genes related to Vancomycin resistance was accomplished using a homologous recombination vector targeting the entire operon (SEQ ID NO: 8). in Brevibacillus laterosporus A8.11 (CBS14109). A synthetic section of DNA was synthesized, corresponding to the genome directly upstream of the Vancomycin resistance genes linked directly to the downstream section, such that the synthetic sequence eliminates all intervening sequence containing the genes (SEQ ID NO: 9; Integrated DNA Technologies, Coralville, Iowa). The synthetic DNA construct was assembled into an integration vector (Leenhouts, K. J., et. al, 1991, Plasmid, 26 (1), 55-66; Maguin, E., et al., 1992. J Bacteriol, 174 (17), 5633-5638). Successful assembly of the synthetic construct into the integration vector was determined by PCR using the primer set primer set A3 (SEQ ID NO: 10) and primer set B3 (SEQ ID NO: 11). The deleted Vancomycin resistance operon sequence was confirmed by DNA sequencing using the same primers (SEQ ID NO: 10 and SEQ ID NO: 11).

Additional genes, potentially related to Vancomycin resistance, were removed using a homologous recombination vector targeting the location within the Brevibacillus laterosporus A8.11 genome (SEQ ID NO: 12). A synthetic section of DNA was synthesized, corresponding to the genome directly upstream of the putative Vancomycin resistance genes linked directly to the downstream section, such that the synthetic sequence eliminates all intervening sequence containing the genes (SEQ ID NO: 13; Integrated DNA Technologies, Coralville, Iowa). Successful assembly of the synthetic construct into the integration vector was determined by PCR using the primer set primer set C (SEQ ID NO: 14) and primer set D (SEQ ID NO: 15). The deleted Vancomycin resistance operon sequence was confirmed by DNA sequencing using the same primers (SEQ ID NO: 14 and SEQ ID NO: 15).

Brevibacillus laterosporus strains also contains genes associated with a virulence factor (Cytolysin). In one aspect, elimination of cytolysin genes is desired, especially for Brevibacillus laterosporus production strains. The cytolysin gene (SEQ ID NO: 16) was removed using a homologous recombination vector targeting its location within the Brevibacillus laterosporus genome. A synthetic section of DNA was synthesized, corresponding to the genome directly upstream of the cytolysin gene linked directly to the downstream section, such that the synthetic sequence eliminates all intervening sequence containing the genes (SEQ ID NO: 17; Integrated DNA Technologies, Coralville, Iowa). The synthetic DNA construct was assembled into an integration vector (Leenhouts, K. J., et. al, 1991, Plasmid, 26 (1), 55-66; Maguin, E., et al., 1992. J Bacteriol, 174 (17), 5633-5638). Successful assembly of the synthetic construct into the integration vector was determined by PCR using the primer set primer set E (SEQ ID NO: 18) and primer set F (SEQ ID NO: 19). The deleted Vancomycin resistance operon sequence was confirmed by DNA sequencing using the same primers (SEQ ID NO: 18 and SEQ ID NO: 19).

Three Vancomycin and cytolysin deletions strains (confirmed by DNA sequencing as described above) were isolated from the last step of the three consecutive knockouts. The three strains were designated Brevibacillus laterosporus ALS311, Brevibacillus laterosporus ALS317, and Brevibacillus laterosporus ALS321, respectively, and deposited at Westerdijk Fungal Biodiversity Institute (WFDB) under number CBS149785, CBS149786, CBS149788, respectively.

A Brevibacillus laterosporus suitable for use in the present invention includes, but is not limited to, a Brevibacillus laterosporus having a 16S ribosomal RNA sequence displaying at least 97.0% sequence similarity to a 16S ribosomal RNA sequence of Brevibacillus laterosporus ALS311 (SEQ ID NO: 1) deposited at Westerdijk Fungal Biodiversity Institute (WFDB) under number CBS149785; a Brevibacillus laterosporus having a 16S ribosomal RNA sequence displaying at least 97.0% sequence similarity to a 16S ribosomal RNA sequence of Brevibacillus laterosporus ALS317 (SEQ ID NO: 1) deposited at Westerdijk Fungal Biodiversity Institute (WFDB) under number CBS149786; and a Brevibacillus laterosporus having a 16S ribosomal RNA sequence displaying at least 97.0% sequence similarity to a 16S ribosomal RNA sequence of Brevibacillus laterosporus ALS321 (SEQ ID NO: 1) deposited at Westerdijk Fungal Biodiversity Institute (WFDB) under number CBS149788.

### Example 2

### Fermentates of Brevibacillus laterosporus

Brevibacillus laterosporus strains were grown in TSB media (1.7% Tryptone, 0.3% Soytone, 0.25% Glucose, 0.5% sodium chloride, 0.25% Potassium phosphate Dibasic) in 250 ml baffled shake-flasks and incubated at 33° C., 250 RPM for 16-18 hours. Following the 16-18-hour incubation, cells were transferred to fresh TSB media with added glucose (10 g/L) or M9 media (0.64% Na₂HPO4-7H₂O, 0.15% KH₂PO4, 0.025% NaCl, 0.05% NH4Cl, 10% glucose) or M9 media+Maltodextrin (0.64% Na₂HPO4-7H₂O, 0.15% KH₂PO4, 0.025% NaCl, 0.05% NH4Cl, 10% Maltodextrin) or M9 media+Maltodextrin+urea (0.64% Na₂HPO4-7H₂O, 0.15% KH₂PO4, 0.025% NaCl, 0.05% NH4Cl, 10% Maltodextrin, 5% urea) in 500 ml bioreactors to an optical density (absorbance at 600 nm), of 0.6-0.7. Cells were grown at 28° C. and 200 RPM, with a continuous air flow of 25 mm/hour. Antifoam (Foamblast® 882, Dystar International) was added 0.04% volume/volume at the start of fermentation and again at 24 hours. Ferment (mixture of culture medium and cells) was collected from bioreactors after 48 hours incubation. Brevibacillus laterosporus strains can be grown in any media that allows for cell growth.

Cells were removed from the ferment by pelleting cells (centrifuge at 4,000 to 8,000×g) and passing the supernatant through a 0.2 μM filter, to obtain an essentially cell-free supernatant. The cell-free supernatant was then assayed for antimicrobial, antioxidant, or surfactant, a foam stabilizer, a foam enhancer activity.

### Example 3

### Biosurfactant Properties of a Multifunctional Fermentate from Brevibacillus laterosporus

Surfactants are wetting agents that reduce the surface tension of a liquid (Berg, J. C. (2010). An introduction to interfaces & colloids: the bridge to nanoscience. World Scientific. pp. 137-147). This example studies the surfactant properties of the Brevibacillus laterosporus fermentate.

The Brevibacillus laterosporus A8.11 fermentate was prepared using the M9+Maltodextrin+urea media solution that included maltodextrin as the carbon source and urea as a nitrogen source (0.64% Na₂HPO4-7H₂O, 0.15% KH₂PO4, 0.025% NaCl, 0.05% NH4Cl, 10% Maltodextrin, 5% urea), as described in Example 2.

The surface tension was measured using the Wilhelmy plate method by KRÜSS Tensiometer (Park, J et al, 2018, Applied Surface Science, 427, 273-280).

As shown in Table 1, the growth media solution (control solution) showed a surface tension that was comparable to the one of water. The fermentate showed a significantly lower surface tension of 36.4 mN·m⁻¹.

TABLE 1
Surface tension measured by KRÜSS Tensiometer

| Samples | Surface Tension (mN · m⁻¹) |
|---|---|
| Water | 72.6 |
| M9 media solution | 70.2 |
| Fermentate solution | 36.4 |

These results indicate that the Brevibacillus laterosporus fermentate has biosurfactant properties to form micelles, and therefore, reduces the surface tension of the aqueous solution. For comparison, anionic surfactants that are commonly found in personal care products have surface tension ranging from 36.4 to 33.7 mN·m⁻¹ at its critical micelle concentration (Trawilska, A et al, 2016, Colloids and Surfaces A: Physicochemical and Engineering Aspects, 506, 114-126).

The micelle particle size of the fermentate was characterized using the Wyatt dynamic light scattering. The fermentate was captured by the resin in HPLC and then extracted using methanol/water 50/50 (v/v) solution. The extracted fermentate was dried to remove methanol and then redissolved in water to make the final concentration at approximately 2 mg/mL. The final fermentate solution was passed through a 0.45 μm syringe filter before characterizing in the dynamic light scattering (repeated for 5 times). The average hydrodynamic radius, representing the micelle particle size, was shown to be 67 nm with a polydispersity of 26%. The measured sub-100 nm particle size distribution further confirmed the surfactant properties of the fermentate to self-assemble into micelles. As a result, the surface tension of the aqueous solution with dissolved fermentate deceased, as shown in Table 1.

### Example 4

### Antioxidant Properties of a Multifunctional Fermentate from Brevibacillus laterosporus

The antioxidant properties of the fermentate from Brevibacillus laterosporus A8.11 in fermentate-dosed formulations were evaluated in serum and hydrogel personal care formulations.

Table 2 shows a xanthan-gum-based serum and Table 3 shows a xanthan-gum-based hydrogel composition. The serum composition was made by heating up the mixture to 75-80° C. and mixed by a homogenizer at approximately 45° C. The hydrogel was made by first sprinkling the xanthan gum powder slowly into a beaker with constant mixing. The mixture was then heated to 75-80° C. and mixed until no evidence of phase separation. The hydrogel was then let cool to room temperature and speed-mixed to remove bubbles. The hydrogel appeared to be transparent.

TABLE 2
Serum Formulation with Xanthan Gum

| Ingredients | INCI Name | WT % |
|---|---|---|
| Water | Water | 82.40 |
| Xanthan gum | Xanthan gum | 0.20 |
| Aristoflex AVC | Ammonium | 0.20 |
| | acryloyldimethyltaurate/ | |
| | vinylpyrrolidone | |
| | copolymer | |
| Hyaluronic acid | Hyaluronic acid | 0.15 |
| Propylene glycol | Propylene glycol | 4.00 |
| Glycerin | Glycerin | 5.00 |
| Disodium EDTA | Disodium EDTA | 0.05 |
| Genencare ® OSMS Pro | Aqua (water), betaine, | 3.00 |
| | proline, inositol, serine | |
| Cremophor ® RH 40 | PEG-40 Hydrogenated | 3.00 |
| | Castor Oil | |
| XIAMETER ™ PMX-200 | Dimethicone | 2.00 |
| Silicone Fluid 100 cSt | | |

TABLE 3
Hydrogel Formulation with Xanthan Gum

| No. | Ingredients | INCI Name | WT % |
|---|---|---|---|
| 1 | Water | Water | 99.5 |
| 2 | Xanthan gum | Xanthan gum | 0.50 |

The Brevibacillus laterosporus fermentate was prepared as described in Example 2, using TSB media (1.7% Tryptone, 0.3% Soytone, 0.25% Glucose, 0.5% sodium chloride, 0.25% Potassium phosphate Dibasic) as the growing solution. The fermentate supernatant was first let flow through an Amicon® Ultra Centrifugal Filters with a size cutoff of 30 kDa. The permeate (which was essentially free of cells) was kept and then let flow through another Amicon® Ultra Centrifugal Filters with a size cutoff of 3 kDa. The retentate was concentrated by 10-fold in volume and stored as the final product (i.e., size is greater than 3 kDa and less than 30 kDa), referring as the concentrated fermentate in the subsequent experiments. The 10-fold concentrated fermentate was dosed separately at 2 weight percent into the serum (Table 2) and hydrogel (Table 3) formulations with xanthan gum.

Antioxidant properties were studied using the activated ABTS assay (Riya, M. P., Antu, K. A., Vinu, T., Chandrakanth, K. C., Anilkumar, K. S., & Raghu, K. G. (2014). An in vitro study reveals nutraceutical properties of Ananas comosus (L.) Merr. var. Mauritius fruit residue beneficial to diabetes. Journal of the Science of Food and Agriculture, 94 (5), 943-950). α-Tocopherol is a type of vitamin E, a commonly known antioxidant. The water-soluble analogue of α-Tocopherol, Trolox, is often used as a reference for characterization the antioxidant properties of a new ingredient in the assay (Reference: “Huang, S. W., Hopia, A., Schwarz, K., Frankel, E. N., & German, J. B. (1996). Antioxidant activity of α-tocopherol and trolox in different lipid substrates: bulk oils vs oil-in-water emulsions. Journal of Agricultural and Food Chemistry, 44 (2), 444-452.”).

A series of Trolox with different concentrations were used as the calibration curve for quantifying the antioxidant properties. The measured absorbance was measured at 405 nm as shown in Table 4.

TABLE 4
Absorbance of the Trolox as a function of concentration
at 405 nm (measured by activated ABTS assay)

| Trolox | Measured |
|---|---|
| (μM) | absorbance |
| 0 | 1.18 |
| 4.7 | 1.16 |
| 9.4 | 1.13 |
| 18.75 | 1.09 |
| 37.5 | 0.97 |
| 75 | 0.77 |
| 150 | 0.38 |

Table 5 summarizes the measured absorbance and the corresponding trolox equivalent (calculated from the Trolox calibration curve) of the fermentate and fermentate-dosed formulations.

TABLE 5
Absorbance of the fermentate and fermentate-dosed formulations
at 405 nm (measured by activated ABTS assay)

| | Measured | Calculated trolox |
|---|---|---|
| Sample | absorbance | equivalent (uM) |
| Fermentate | 0.23 | 177 |
| Serum (10X diluted) | 1.21 | ~0 |
| Serum dosed with 2 wt % | 1.09 | 17 |
| fermentate (10X diluted) | | |
| Hydrogel (10X diluted) | 1.19 | ~0 |
| Hydrogel dosed with 2 | 1.01 | 32 |
| wt % fermentate (10X | | |
| diluted) | | |

The fermentate showed strong intrinsic antioxidant proprieties, equivalent to 177 uM of the trolox. The serum and hydrogel formulations were diluted at 10-fold while running the activated ABTS assay to ensure the formulations have no contribution to the antioxidant properties. As shown in Table 7, both formulations showed no antioxidant properties (i.e., ˜0 uM trolox equivalent) at 10-fold dilution. However, the 2 wt % fermentate-dosed serum and hydrogel formulations at 10-fold dilution showed significant antioxidant properties, equivalent to 17 and 32 uM trolox, respectively.

### Example 5

### Antimicrobial Enhancing Properties of a Multifunctional Fermentate from Brevibacillus laterosporus in Different Personal Care Formulations

The enhancing activity of soft preservatives by Brevibacillus laterosporus fermentate in personal care-dosed formulations was evaluated using a modification of the Personal Care Products Council (PCPC) protocol. (Personal Care Products Council (PCPC—formerly the Cosmetic, Toiletry and Fragrance Association); CTFA Microbiology Guidelines 2007).

The Brevibacillus laterosporus fermentate was prepared using the M9 media (0.64% Na₂HPO4-7H₂O, 0.15% KH₂PO4, 0.025% NaCl, 0.05% NH4Cl, 10% glucose), as described in example 2. The produced fermentate was centrifuged and the cell free supernatant solution was first let flow through an Amicon® Ultra Centrifugal Filters with a size cutoff of 30 kDa. The permeate was kept and then let flow through another Amicon® Ultra Centrifugal Filters with a size cutoff of 3 kDa. The retentate was concentrated by 10-fold in volume and stored as the final product (i.e., size is greater than 3 kDa and less than 30 kDa), referring as the concentrated fermentate in the subsequent experiments.

To test the efficacy of Brevibacillus laterosporus fermentate to enhance the activity of a soft preservative such as phenoxyetanol in multiple personal care formulations, the concentrated fermentate and phenoxyethanol were dosed at room temperature into the three different personal care formulations, including a cream with xanthan gum (Table 6), a lotion with carbomer (Table 7), and a serum formulation with xanthan gum (Table 8).

Table 6 shows an exemplary formulation for xanthan-gum-based anionic cream composition. Xanthan gum is one of the most widely used natural rheological modifiers in personal care products. The formulation below was made by heating the aqueous (A) and oil (B) phases separately to 75-80° C. The oil phase was then poured into the aqueous phase and mixed by a homogenizer at approximately 45° C. The formed oil-in-water emulsion was let cool to room temperature before adjusting its pH to 5.0-5.5.

TABLE 6
Cream Formulation with Xanthan Gum

| Phase | Ingredients | INCI Name | WT % |
|---|---|---|---|
| A | Water | Water | 79.7 |
| | Keltrol CG-RD | Xanthan Gum | 0.3 |
| | Propylene Glycol | Propylene Glycol | 4.0 |
| B | Petrolatum | Petrolatum | 2.0 |
| | HallStar ® IPM NF | Isopropyl Myristate | 3.0 |
| | Sensolene (Hallstar) | Ethylhexyl Olivate | 4.0 |
| | HallStar ® GMS Pure | Glyceryl Stearate | 2.0 |
| | Stearic Acid | Stearic Acid | 3.0 |
| | Cosmowax ™ P | Cetearyl Alcohol and | 2.0 |
| | | Ceteareth-22 | |

Table 7 shows an exemplary formulation for carbomer-based anionic lotion compositions. Carbomers are a commonly used synthetic rheological modifiers in personal care products. The formulation below was made by heating the aqueous (A) and oil (B) phases separately to 75-80° C. The oil phase was then poured into the aqueous phase and mixed by a homogenizer at approximately 45° C. The formed oil-in-water emulsion was let cool to room temperature. Triethanolamine was used as the crosslinker to adjust the final pH to 6.0-6.5.

TABLE 7
Lotion Formulation with Carbomer

| Phase | Ingredients | INCI Name | WT % |
|---|---|---|---|
| A | Water | Water | 88.8 |
| | Glycerin | Glycerin | 2.0 |
| | Carbopol 940 (Lubrizol) | Carbomer | 0.2 |
| B | Cream Maker CA-20 | Cetearyl Alcohol and | 2.0 |
| | | Ceteareth-20 | |
| | Rita GMS (Rita Corp) | Glyceryl Stearate | 1.0 |
| | Isopropyl Myristate | Isopropyl Myristate | 1.0 |
| | Shea Butter | Shea Butter | 1.0 |
| | Caprylic/Capric | Caprylic/Capric | 2.0 |
| | Triglyceride | Triglyceride | |
| | Isohexadecane | Isohexadecane | 1.0 |
| | Silicone Oil Xiameter | Dimethicone | 1.0 |
| | PMX-200 5 cSt | | |

Table 8 shows a xanthan-gum-based serum. The serum composition was made by adding all the ingredients into a 2 liter beaker and heating up all the ingredient mixture (Table 8) to 75-80° C. and mixed by a homogenizer at approximately 45° C. The final product was let cool to room temperature before dosing any soft preservative or fermentate.

TABLE 8
Serum Formulation with Xanthan Gum

| Ingredients | INCI Name | WT % |
|---|---|---|
| Water | Water | 82.40 |
| Xanthan gum | Xanthan gum | 0.20 |
| Aristoflex AVC | Ammonium | 0.20 |
| | acryloyldimethyltaurate/ | |
| | vinylpyrrolidone | |
| | copolymer | |
| Hyaluronic acid | Hyaluronic acid | 0.15 |
| Propylene glycol | Propylene glycol | 4.00 |
| Glycerin | Glycerin | 5.00 |
| Disodium EDTA | Disodium EDTA | 0.05 |
| Genencare ® OSMS Pro | Aqua (water), betaine, | 3.00 |
| | proline, inositol, serine | |
| Cremophor ® RH 40 | PEG-40 Hydrogenated | 3.00 |
| | Castor Oil | |
| XIAMETER ™ PMX-200 | Dimethicone | 2.00 |
| Silicone Fluid 100 cSt | | |

Dosed formulations comprising several different doses (wt %) of fermentate and soft preservative (POE) in a personal care formulation are shown in Tables 9, 10 and 11.

TABLE 9
Dosing of fermentate and soft preservative
in a personal care cream formulation.

| | wt % Cream | wt % | wt % |
|---|---|---|---|
| Formulation name | (Table 6) | fermentate | POE |
| Blank cream | 100 | 0 | 0 |
| Phenoxyethanol (POE) 0.3% | 99.7 | 0 | 0.3 |
| Fermentate 1% | 99 | 1 | 0 |
| Fermentate 2.5% | 97.5 | 2.5 | 0 |
| Fermentate 5.0 | 95 | 5.0 | 0 |
| POE 0.3% + Fermentate 1% | 98.7 | 1 | 0.3 |
| POE 0.3% + Fermentate 2.5% | 97.2 | 2.5 | 0.3 |
| POE 0.3% + Fermentate 5.0 | 94.7 | 5.0 | 0.3 |

TABLE 10
Dosing of fermentate and soft preservative
in a personal care lotion formulation.

| | wt % Lotion | wt % | |
|---|---|---|---|
| Formulation name | (Table 7) | fermentate | wt % POE |
| Blank lotion | 100 | 0 | 0 |
| Phenoxyethanol (POE) 0.4% | 99.6 | 0 | 0.4 |
| Fermentate 1% | 99 | 1 | 0 |
| Fermentate 2.5% | 97.5 | 2.5 | 0 |
| Fermentate 5.0 | 95 | 5.0 | 0 |
| POE 0.4% + Fermentate 1% | 98.6 | 1 | 0.4 |
| POE 0.4% + Fermentate 2.5% | 97.1 | 2.5 | 0.4 |

TABLE 11
Dosing of fermentate and soft preservative
in a personal care serum formulation.

| | wt % Serum | wt % | |
|---|---|---|---|
| Formulation name | (Table 9) | fermentate | wt % POE |
| Blank serum | 100 | 0 | 0 |
| Phenoxyethanol (POE) 0.4% | 99.6 | 0 | 0.4 |
| Phenoxyethanol (POE) 0.5% | 99.5 | 0 | 0.5 |
| Fermentate 5.0 | 95 | 5.0 | 0 |
| POE 0.4% + Fermentate 2.5% | 97.1 | 2.5 | 0.4 |
| POE 0.5% + Fermentate 2.5% | 97.1 | 2.5 | 0.4 |

The dosed formulations (see Tables 9-11 for the dosing levels) were speed-mixed to ensure a homogeneity. The microbial preservative efficacy test (challenge test) was conducted using a four week, two-cycle microbial preservative efficacy test at 25° C. to determine the preservative efficacy of each treatment.

After dosing, the samples were divided into two separate, five gram aliquots. One aliquot was inoculated with 50 μl of a diluted bacterial pool, the second aliquot was inoculated with 50 μl of a diluted fungal pool as described below. Unpreserved product samples were included in our test as a growth control.

A mixed bacterial inoculum was prepared using 24-hour cultures of the test bacteria (Table 12) grown in trypticase soy broth (TSB). Equal volumes of the bacterial test strains were combined and diluted one to ten in phosphate buffer to obtain an inoculum of approximately 5×10⁷ to 5×10⁸ colony forming units per ml (cfu/ml). The test samples were inoculated with 1% of the mixed bacterial inoculum. A mixed fungal inoculum was prepared using cell suspensions of the yeast Candida albicans ATCC #10231 and the mold Aspergillus brasiliensis ATCC #16404 in phosphate buffer. Equal volumes of the fungal test strains were combined and diluted one to ten with phosphate buffer to obtain an inoculum of approximately 5×10⁶ to 5×10⁷ cfu/ml. The test samples were inoculated with 1% of the mixed fungal inoculum.

TABLE 12
Consumer Products Mixed Bacterial Inoculum

| Microorganism | ATCC Number | Type |
|---|---|---|
| Burkholderia cepacia | 25416 | Gram-Negative |
| | | Non-fermenter |
| Enterobacter gergoviae | 33028 | Gram-Negative Fermenter |
| Escherichia coli | 8739 | Gram-Negative Fermenter |
| Klebsiella pneumoniae | 13883 | Gram-Negative Fermenter |
| Pseudomonas aeruginosa | 9027 | Gram-Negative |
| | | Non-fermenter |
| Pseudomonas aeruginosa | 15442 | Gram-Negative |
| | | Non-fermenter |
| Pseudomonas putida | 49128 | Gram-Negative |
| | | Non-fermenter |
| Staphylococcus aureus | 6538 | Gram-Positive |
| Staphylococcus epidermidis | 12228 | Gram-Positive |

The samples were subjected to microbial challenge at time zero and inoculated a second time after seven days. The number of microorganisms added to each sample was determined by a standard Most Probable Number (MPN) determination in Trypticase Soy Broth (TSB) for bacteria and Potato Dextrose Broth (PDB) for fungi. Samples were incubated at 25° C. for the test duration of four weeks. Samples were monitored for bacterial and fungal contamination after 2, 7, 14, 21, and 28 days. Samples challenged with bacteria were streak-plated onto Trypticase Soy Agar (TSA) and incubated at 30° C. for 24 hours. Samples challenged with fungi were streak-plated onto Potato Dextrose Agar (PDA) and incubated at 25° C. for 7 days. After incubation, plates were given a Growth Rating to determine the colony forming units per gram (CFU/g) present in each test sample at the testing time point. Table 13 represents the growth score.

TABLE 13
Growth score used to determine CFU/mL in test samples.

| Growth | Description of | | Approximate |
|---|---|---|---|
| Rating | Microbial Growth | Plating Results | cfu/g |
| 0 | No contamination | No detectable | <10 |
| | | growth | |
| 1 | Trace contamination | 1 to 9 colonies | 1 × 10¹ to 9 × 10¹ |
| 2 | Light contamination | 10 to 99 colonies | 1 × 10² to 9.9 × 10² |
| 3 | Moderate | 100 to 1,000 | 1 × 10³ to 9.9 × 10³ |
| | contamination | colonies | |
| 4 | Heavy contamination | >1,000 or smear | >1 × 10⁴ |

A formulation was scored as “Pass” when the formulation obtained a growth rating score caused by bacteria of 1 or less at days 21-28 of the testing period.

A formulation was scored as “Pass” when the formulation obtained a growth rating score caused by fungi of 2 or less at days 21-28 of the testing period.

A formulation was scored as “Fail” when the formulation obtained a growth rating score caused by bacteria of 2 or more at days 21-28 of the testing period.

A formulation was scored as “Fail” when the formulation obtained a growth rating score caused by fungi of more than 2 at days 21-28 of the testing period.

Tables 14 A-C, Table 15 A-C and Table 16 A-C summarize the challenge test data for the three fermentate-dosed formulations at different dosing level.

TABLE 14 A
Bacteria Challenge test data in the cream
formulation comprising xanthan gum

| | Bacteria growth rating | | | | | Bacteria |
|---|---|---|---|---|---|---|
| Sample and dosing (wt %) | D 2 | D 7 | D 14 | D 21 | D 28 | Result |
| Blank cream | 4 | 4 | 4 | 4 | 3 | Fail |
| Phenoxyethanol (POE) | 4 | 2 | 3 | 2 | 1 | Fail |
| 0.3% | | | | | | |
| Fermentate 1.0% | 4 | 4 | 4 | 4 | 3 | Fail |
| Fermentate 2.5% | 4 | 4 | 4 | 4 | 3 | Fail |
| Fermentate 5.0% | 4 | 4 | 4 | 4 | 3 | Fail |
| POE 0.3% + Fermentate | 4 | 3 | 3 | 2 | 2 | Fail |
| 1.0% | | | | | | |
| POE 0.3% + Fermentate | 3 | 1 | 0 | 0 | 0 | Pass |
| 2.5% | | | | | | |
| POE 0.3% + Fermentate | 2 | 0 | 0 | 0 | 1 | Pass |
| 5.0% | | | | | | |

TABLE 14 B
Fungi Challenge test data in the cream
formulation comprising xanthan gum

| | Fungi growth rating | | | | | Fungi |
|---|---|---|---|---|---|---|
| Sample and dosing (wt %) | D 2 | D 7 | D 14 | D 21 | D 28 | Result |
| Blank cream | 4 | 4 | 4 | 3 | 3 | Fail |
| Phenoxyethanol (POE) | 3 | 3 | 4 | 3 | 3 | Fail |
| 0.3% | | | | | | |
| Fermentate 1.0% | 4 | 4 | 4 | 3 | 3 | Fail |
| Fermentate 2.5% | 4 | 4 | 3 | 3 | 3 | Fail |
| Fermentate 5.0% | 3 | 3 | 3 | 3 | 3 | Fail |
| POE 0.3% + Fermentate | 3 | 2 | 4 | 3 | 3 | Fail |
| 1.0% | | | | | | |
| POE 0.3% + Fermentate | 3 | 3 | 3 | 3 | 3 | Fail |
| 2.5% | | | | | | |
| POE 0.3% + Fermentate | 3 | 2 | 3 | 2 | 1 | Pass |
| 5.0% | | | | | | |

TABLE 14 C
Challenge test data summary in the cream
formulation comprising xanthan gum

| | Bacteria | Fungi | Overall |
|---|---|---|---|
| | Result | Result | Result |
| Sample and dosing (wt %) | | | |
| Blank cream | Fail | Fail | Fail |
| Phenoxyethanol (POE) 0.3% | Fail | Fail | Fail |
| Fermentate 1.0% | Fail | Fail | Fail |
| Fermentate 2.5% | Fail | Fail | Fail |
| Fermentate 5.0% | Fail | Fail | Fail |
| POE 0.3% + Fermentate 1.0% | Fail | Fail | Fail |
| POE 0.3% + Fermentate 2.5% | Pass | Fail | Fail |
| POE 0.3% + Fermentate 5.0% | Pass | Pass | Pass |

TABLE 15 A
Bacteria Challenge test data in the lotion
formulation comprising carbomer gum

| | Bacteria growth rating | | | | | Bacteria |
|---|---|---|---|---|---|---|
| Sample and dosing (wt %) | D 2 | D 7 | D 14 | D 21 | D 28 | Result |
| Blank lotion | 4 | 4 | 4 | 4 | 3 | Fail |
| Phenoxyethanol (POE) | 4 | 3 | 3 | 3 | 1 | Fail |
| 0.4% | | | | | | |
| Ferment 1.0% | 4 | 4 | 4 | 4 | 3 | Fail |
| Ferment 2.5% | 4 | 4 | 4 | 4 | 3 | Fail |
| Ferment 5.0% | 4 | 4 | 4 | 4 | 3 | Fail |
| POE 0.4% + Ferment 1.0% | 3 | 0 | 1 | 0 | 0 | Pass |
| POE 0.4% + Ferment 2.5% | 3 | 0 | 2 | 1 | 0 | Pass |

TABLE 15 B
Fungi Challenge test data in the lotion
formulation comprising carbomer gum

| | Fungi growth rating | | | | | Fungi |
|---|---|---|---|---|---|---|
| Sample and dosing (wt %) | D 2 | D 7 | D 14 | D 21 | D 28 | Result |
| Blank lotion | 4 | 4 | 4 | 3 | 3 | Fail |
| Phenoxyethanol (POE) | 3 | 2 | 2 | 1 | 1 | Pass |
| 0.4% | | | | | | |
| Ferment 1.0% | 4 | 4 | 3 | 3 | 2 | Fail |
| Ferment 2.5% | 4 | 4 | 2 | 3 | 2 | Fail |
| Ferment 5.0% | 4 | 3 | 2 | 2 | 2 | Pass |
| POE 0.4% + Ferment 1.0% | 3 | 2 | 3 | 2 | 1 | Pass |
| POE 0.4% + Ferment 2.5% | 3 | 2 | 3 | 2 | 1 | Pass |

TABLE 15 C
Challenge test data summary in the
lotion formulation with carbomer

| | Bacteria | Fungi | Overall |
|---|---|---|---|
| | Result | Result | Result |
| Sample and dosing (wt %) | | | |
| Blank lotion | Fail | Fail | Fail |
| Phenoxyethanol (POE) 0.4% | Fail | Pass | Fail |
| Ferment 1.0% | Fail | Fail | Fail |
| Ferment 2.5% | Fail | Fail | Fail |
| Ferment 5.0% | Fail | Pass | Fail |
| POE 0.4% + Ferment 1.0% | Pass | Pass | Pass |
| POE 0.4% + Ferment 2.5% | Pass | Pass | Pass |

TABLE 16 A
Bacteria Challenge test data in the serum
formulation comprising xantham gum

| | Bacteria growth rating | | | | | Bacteria |
|---|---|---|---|---|---|---|
| Sample and dosing (wt %) | D 2 | D 7 | D 14 | D 21 | D 28 | Result |
| Blank serum | 4 | 4 | 4 | 4 | 2 | Fail |
| Phenoxyethanol (POE) | 4 | 4 | 3 | 3 | 0 | Fail |
| 0.4% | | | | | | |
| POE 0.5% | 4 | 4 | 3 | 2 | 0 | Fail |
| Ferment 5.0% | 4 | 3 | 4 | 4 | 2 | Fail |
| POE 0.4% + Ferment 2.5% | 4 | 3 | 2 | 1 | 0 | Pass |
| POE 0.5% + Ferment 2.5% | 3 | 2 | 1 | 1 | 0 | Pass |

TABLE 16 B
Fungi Challenge test data in the serum
formulation comprising xantham gum

| | Fungi growth rating | | | | | Fungi |
|---|---|---|---|---|---|---|
| Sample and dosing (wt %) | D 2 | D 7 | D 14 | D 21 | D 28 | Result |
| Blank serum | 4 | 3 | 4 | 3 | 3 | Fail |
| Phenoxyethanol (POE) | 3 | 2 | 3 | 2 | 1 | Pass |
| 0.4% | | | | | | |
| POE 0.5% | 3 | 2 | 3 | 1 | 0 | Pass |
| Ferment 5.0% | 4 | 3 | 4 | 2 | 3 | Fail |
| POE 0.4% + Ferment 2.5% | 3 | 2 | 2 | 2 | 1 | Pass |
| POE 0.5% + Ferment 2.5% | 3 | 2 | 3 | 1 | 0 | Pass |

TABLE 16C
Challenge test data summary in the
serum formulation with xanthan gum

| | Bacteria | Fungi | Test |
|---|---|---|---|
| | Result | Result | Result |
| Sample and dosing (wt %) | | | |
| Blank serum | Fail | Fail | Fail |
| Phenoxyethanol (POE) 0.4% | Fail | Pass | Fail |
| POE 0.5% | Fail | Pass | Fail |
| Ferment 5.0% | Fail | Fail | Fail |
| POE 0.4% + Ferment 2.5% | Pass | Pass | Pass |
| POE 0.5% + Ferment 2.5% | Pass | Pass | Pass |

As shown in Table 14 A-C, Table 15 A-C and Table 16 A-C, none of the formulations passed the challenge test when dosed with phenoxyethanol or ferment alone. When dosed with a combination of the phenoxyethanol and ferment, these formulations showed antimicrobial properties against both bacteria and fungi. This example clearly demonstrates that in all cases, the fermentate was able to enhance the efficacy of the soft preservative phenoxyethanol so that a preservation was obtained at low levels of phenoxyethanol (0.3-0.5%). Formulation containing only phenoxyethanol at these low levels (0.3-0.5%) clearly were not preserved. As such, the Brevibacillus laterosporus fermentate act as an enhancer when combined with a commonly used soft preservative phenoxyethanol, to further improve the antimicrobial properties of the formulations.

### Example 6

### Antimicrobial Activity of Brevibacillus laterosporus Fermentate

Antimicrobial activity of the Brevibacillus laterosporus fermentate was also demonstrated with assays performed in 96-well plates by adding ferment, or fractions thereof, adjusted to pH 6.6-7.0 to TSB media containing microbial test strains which were present at an initial optical density of 0.05 (absorbance at 600 nm). Each cell-free supernatant (ferment) was tested in replicates of four separate wells. Test strains included Escherichia coli, ATCC 8739, Staphylococcus epidermidis, ATCC 12228 and Candida albicans, ATCC 10231.

The 96-well plates were incubated under aerobic conditions at 33° C. for 18-20 hours. Following incubation, the optical density was determined for each well using a microplate reader (Molecular Devices). Following incubation, an average of four replicate wells with no increase in turbidity, indicating no growth of the test strain, were considered positive for antimicrobial activity. Average turbidity for wells which contained no added ferment were used as controls. The data in Table 17 demonstrates growth for all three test strains with no added ferment. Antimicrobial activity was demonstrated, showing no growth for test strains in wells containing the added ferment.

TABLE 17
Average optical density (A600nm) for four replicate wells. Control
samples contain microbial test cells with added TSB media. Ferment
was tested at 20% volume with indicated target strain.

| | Escherichia coli | Staphylococcus epidermidis | Candida albicans |
|---|---|---|---|
| | ATCC 8739 | ATCC 12228 | ATCC 10231 |
| Control | 0.611 | 0.788 | 0.622 |
| Ferment | 0.048 | 0.040 | 0.042 |

As shown in Table 17 control samples indicated growth of cells with a much higher optical density compared to cells containing ferment, which did not grow.

### Example 7

### Compatibility of Brevibacillus laterosporus Fermentate with Anionic Components of Personal Care Formulations

Anionic and cationic components are not readily compatible with each other in formulations. Evidence of incompatibility ranges from visually noticed precipitation and formulation separation to loss of key activity of the ingredients. Cationic components of the growth media can interfere with the stability of the fermentate. Turbidity in the growth media can be particularly problematic as it can interfere reading optical density of cell cultures, or measures of turbidity. To alleviate this issue, an anionic water treatment polymer designed to bind to magnesium and calcium ions (such as the polyacrylate polymer in Acumer 4200) can be added to the formulation making the formulation anion.

Fermentates of Brevibacillus laterosporus strain G2 were prepared as described in Example 2, using a rich media like tryptic soy broth (TSB). The efficacy of the Brevibacillus laterosporus fermentate to inhibit the growth of S. aureus was studied.

with and without the addition of an anionic compound such as Acumner 4200, to determine the compatibility of the fermentate in an anionic environment. The polyacrylate polymer in Acumer 4200 remained highly water soluble, even in the presence of relatively large abundance of cationic compounds.

The compatibility of the Brevibacillus laterosporus fermentate with anionic compounds was compared to the compatibility of fermentates from other microorganisms with anionic components. Fermentates of Micrococcus yunnanensis str. YIM 65004 and Bacillus subtilis subsp. spizizenii str. NRRL B were prepared in a similar fashion as the Brevibacillus laterosporus strain G2 fermentates.

All three fermentates were able to inhibit to greater than 80% Staphylococcus aureus growth in unsupplemented TSB growth media (Table 20). The fermentates were tested for retained efficacy in growth media containing the anionic polyacrylate polymer Acumer 4200. The percent inhibition was determined by taking the OD600 reading (looking for turbidity) and comparing the unmodified control to samples where 20% v/v of the fermentate of the specified organism was added to the growth media. A negative number indicates that rather than inhibiting the growth of S. aureus, there was an increase in growth relative to the control sample. The taxonomic designation was determined by 16S sequencing.

TABLE 18
Percent inhibition of Staphylococcus aureus by microbial
fermentates in the absence or presence of anionic compounds.

| | | % Inhibition |
|---|---|---|
| | % Inhibition | S. aureus in |
| Taxonomic Designation (16s) | S. aureus_6538 | presence Acumer 4200 |
| Micrococcus yunnanensis str. | 99.6 | −7.3 |
| YIM 65004 | | |
| Bacillus subtilis subsp. | 98.7 | −82.8 |
| spizizenii str. NRRL B-23049 | | |
| Brevibacillus laterosporus G2 | 99.8 | 76.1 |

Table 18 shows that the fermentate of Brevibacillus laterosporus G2 was able to suppress growth of S. aureus in an anionic environment while Micrococcus yunnanensis and Bacillus subtilis subspecies were not able to suppress growth (actually their growth was stimulated as is evident from negative % inhibition), clearly indicating that the Brevibacillus laterosporus fermentate activity is stable in an anionic environment.

The ability of Polylysine, a known cationic peptide-based food preservative, to suppress the growth of Pseudomonas aeruginosa in the presence and absence of the anionic polymer Acumer 4200 was also evaluated. The test was conducted in scintillation vials inoculated with a 1:100 dilution from an overnight of Pseudomonas aeruginosa (ATCC #15442) allowed to incubate at 37 C overnight. The readout was visual with turbidity as an indicator of Pseudomonas growth.

TABLE 19
Ability of Polylysine to control Pseudomonas aeruginosa
in the presence and absence of anionic polymer Acumer 4200.

| Vial Contents | |
|---|---|
| (Inoculated with Pseudomonas) | Visual Inspection |
| TSB growth medium, polylysine | Clear (polylysine prevented growth) |
| TSB growth Medium, Acumer | Turbid |
| 4200 | (growth in the presence of Acumer |
| | 4200) |
| TSB growth medium, Acumer | Turbid |
| 4200, polylysine | (the interaction between the polylysine |
| | and the Acumer 4200 prevented the |
| | antimicrobial activity of the polylysine) |

Results from Table 19 indicates that while polylysine was able to suppress microbial growth in the absence of an anionic polymer, it does not function as an antimicrobial in the presence of the anionic surfactant Acumen 4200.

Personal care formulations often contain thickeners and/or rheology modifiers such as xanthan gum, which is an anionic ingredient. Due to the cationic nature of many antimicrobial peptides, they are often not compatible with formulations including xanthan gum. Xanthan gum used in personal care can give a close to optically clear formulation allowing for continued use of optical density/turbidity (OD600) as the readout in assays. Although it is a rheology modifier, use levels of 0.25% are readily pipetable making testing simpler in smaller quantities, yet relevant to application dose levels.

The efficacy of Brevibacillus laterosporus fermentate A8.11 to reduce the growth of Candida bacteria in the presence and absence of anionic ingredients, represented by xantham gum is shown in Table 22. Candida was inoculated in TSB growth media with fermentate produced from a bioreactor (M9 growth media) in successive dilutions from 20%-2% v/v fermentate. The control growth media was 50% TSB growth medium diluted with water and the xanthan gum supplemented media was 50% TSB growth medium diluted with 0.5% xanthan gum in water. The final concentration of the xanthan gum in the TSB media is 0.25%. The test conditions were incubated overnight at 30 C before being read for absorbance at 600 nm (OD600). Higher OD600 values indicate higher turbidity, which is a proxy for higher levels of microbial growth.

TABLE 20
Candida growth in media comprising Brevibacillus laterosporus
fermentate supplemented with or without xantham gum. (Pass =
growth inhibition. Fail = inadequate growth inhibition).

| Percent | OD 600 | | OD600 | |
|---|---|---|---|---|
| Fermentate | Control Growth Media | | Xanthan gum Supplemented media | |
| 20 | 0.04195 | Pass | 0.112525 | Pass |
| 18 | 0.042725 | Pass | 0.099725 | Pass |
| 16 | 0.0588 | Pass | 0.137025 | Pass |
| 14 | 0.094725 | Pass | 0.148 | Pass |
| 12 | 0.092025 | Pass | 0.27835 | Fail |
| 10 | 0.176525 | Fail | 0.312425 | Fail |
| 8 | 0.1324 | Fail | 0.485775 | Fail |
| 6 | 0.398925 | Fail | 0.482625 | Fail |
| 4 | 0.256275 | Fail | 0.95535 | Fail |
| 2 | 0.09285 | Fail | 0.889675 | Fail |

An OD600 of less than 0.16 was considered to be controlling the desired microorganism. As shown in Table 20, 12% of Brevibacillus fermentate added to the control growth media or 14% added to the xanthan gum supplemented media allowed for the control of Candida growth. The control levels of 12% and 14% are considered within the error of the experiment, meaning that the activity in the anionic based media containing xanthan gum is equivalent to the control growth media. There was no loss of activity from the fermentate when an anionic component is added.

### Example 8

### Blue Light Filtration and UV Light Filtration Properties of a Multifunctional Fermentate from Brevibacillus laterosporus

The Blue light filtration and UV light filtration properties of a Brevibacillus laterosporus A8.11 fermentate were evaluated.

Blue light and UV light filters are increasingly of interest in personal care formulation for their anti-pollution properties and their sunscreen attributes. Effective blue light filters can also reduce skin damages from long-term exposure to visible light. Blue light filters are designed to absorb visible light in the wavelengths of 400-440 nm. UVA filters absorb light in the wavelengths of 315-400, while UVB absorbs light in the wavelengths 280-315.

Two types of Brevibacillus laterosporus fermentates were produced, one originated from Brevibacillus laterosporus A8.11 grown in a minimal growth medium and one from Brevibacillus laterosporus A8.11 grown in a rich growth medium. The two types of growth media were chosen to determine if growth conditions influenced the production of metabolites with blue light or UV light filtration.

The minimal growth medium was composed of the M9+Maltodextran media described in Example 2 supplemented with 5 g/L urea. The fermentate was produced by inoculating the growth media with 10% of a culture prepared in TSB. TSB is a rich growth media described in Example 2. A control for the minimal medium was composed of medium was composed of the supplemented M9 media with 10% TSB. The supplemented M9 media control contains the addition of 10% TSB to account for any light absorbance from the TSB (TSB can have a yellow or orange hue).

The rich growth medium media was composed as described in Example 2 plus the addition of 10 g/L Maltodextran. The fermentate was produced by inoculating the growth media with 10% of a culture prepared in TSB. A control for the rich medium was composed of the supplemented TSB.

The fermentates were analyzed for their ability to absorb light at wavelengths from 300-440 nM. For analysis, fermentates and control media were placed in a clear, flat bottomed 96 well plate with at least 3 wells separating the samples.

TABLE 21
Absorbance reading at each wavelength for a control minimal growth
media and the fermentate produced from a minimal growth medium.

| | | Absorbance of | | |
|---|---|---|---|---|
| | Absorbance of | Ferment | % Increase in | |
| | minimal | originated from | Absorbance | |
| Wavelength | Control | minimal growth | from | Type of |
| (nM) | medium | media | Fermentate | Light |
| 300 | 0.6085 | 1.081 | 178 | UVB |
| 310 | 0.3945 | 0.694 | 176 | UVB |
| 320 | 0.319 | 0.566 | 177 | UVA |
| 330 | 0.2565 | 0.465 | 181 | UVA |
| 340 | 0.211 | 0.3885 | 184 | UVA |
| 350 | 0.175 | 0.33 | 189 | UVA |
| 360 | 0.1395 | 0.279 | 200 | UVA |
| 370 | 0.1135 | 0.2475 | 218 | UVA |
| 380 | 0.0855 | 0.215 | 251 | UVA |
| 390 | 0.0645 | 0.1895 | 294 | UVA |
| 400 | 0.052 | 0.168 | 323 | Blue Light |
| 410 | 0.0435 | 0.146 | 336 | Blue Light |
| 420 | 0.0375 | 0.125 | 333 | Blue Light |
| 430 | 0.0345 | 0.103 | 299 | Blue Light |
| 440 | 0.032 | 0.0885 | 277 | Blue Light |

The data in Table 21 indicates that the light absorbance from the fermentate grown in a minimal growth medium increased between 178-336% when compared to the absorbance of a control medium. This increased absorbance is representative of an increased light filtration at both the UVA/UVB light and the Blue Light level.

TABLE 22
Absorbance reading at each wavelength for a control rich growth
medium and fermentate produced from said rich growth medium.

| | | absorbance of | | |
|---|---|---|---|---|
| | | ferment | % Increase | |
| | absorbance of | originated | absorbance | |
| | rich medium | from rich | from | Type of |
| Wavelength | control | growth media | Fermentate | light |
| 300 | 2.601 | 3.973 | 153 | UVB |
| 310 | 1.8235 | 2.783 | 153 | UVB |
| 320 | 1.5165 | 2.222 | 147 | UVA |
| 330 | 1.272 | 1.838 | 144 | UVA |
| 340 | 1.095 | 1.528 | 140 | UVA |
| 350 | 0.931 | 1.251 | 134 | UVA |
| 360 | 0.7545 | 0.9955 | 132 | UVA |
| 370 | 0.6155 | 0.826 | 134 | UVA |
| 380 | 0.4765 | 0.6655 | 140 | UVA |
| 390 | 0.366 | 0.539 | 147 | UVA |
| 400 | 0.281 | 0.439 | 156 | Blue Light |
| 410 | 0.2215 | 0.361 | 163 | Blue Light |
| 420 | 0.184 | 0.2975 | 162 | Blue Light |
| 430 | 0.152 | 0.24 | 158 | Blue Light |
| 440 | 0.133 | 0.203 | 153 | Blue Light |

The data in Table 22 indicates that the light absorbance from the fermentate grown in a minimal growth medium increased between 132-163% when compared to the absorbance of a control medium. This increased absorbance is representative of an increased light filtration at both the UVA/UVB light and the Blue Light level.

Taken together, fermentates produced from both the rich and minimal medias showed an increase in blue light, UVA, and UVB absorbance.

### Example 9

### Identification of Active Molecules Present in Brevibacillus laterosporus Fermentate

Fermentates of Brevibacillus laterosporus strain A8.11 were prepared as described in Example 2, either using a rich media like tryptic soy broth (TSB) or a minimal media like M9, either supplemented with additional nutrients or without additional supplementation. Minimal media allow for an easier analytical investigation to determine active molecules compared to rich media, while rich media may allow for the production of higher quantities of desired actives.

To allow for HPLC analysis of the fermentate, salts were removed from the fermentate. To remove salts, the sample was concentrated over a 3 KD molecular weight cut off filter with the flow through discarded. Small molecules and salts remain in the flowthrough and can be removed this way. In other cases, a smaller volume of sample containing the salts was run on the HPLC column.

A Prep HPLC is a version of an analytical HPLC that uses much larger column sizes with the goal of recovering the material after separation. The Prep HPLC was run multiple times to obtain optimal separation of desired compounds. In all cases the prep HPLC column was a C18 column and the mobile phase was a mixture of water and acetonitrile, both of which were acidified with formic acid. Formic acid was chosen so that it would be compatible with future mass spectrometry work used to identify the active molecules. Additionally, all methods started with a low concentration of acetonitrile, ramped up to 90 or 95% acetonitrile and then returned to initial starting concentrations of water and acetonitrile. Over the course of the HPLC run, up to 120 fractions were collected containing the mobile phase and eluted compounds. Fractions varied in volume from 8-10 mL. At the conclusion of the prep HPLC run, the fractions were dried completely. In some cases, the fractions were sampled immediately after running and before drying to be run on a corresponding analytical HPLC using a longer method. The analytical HPLC method similarly used water and acetonitrile as the mobile phase but was acidified with trifluoroacetic acid to give better peak shape; these samples were sent to waste and would never be used for mass spec analysis. The purpose of running the analytical HPLC was to tie fractions from the prep HPLC to common peaks observed on the analytical HPLC.

After the prep HPLC fractions were dried, they were dissolved in 1 mL of water and arrayed into microtiter plates. The microtiter plates were used to assay the antimicrobial activity of compounds in each of the fractions against Staphylococcus aureus (ATCC #6538), Pseudomonas aeruginosa (ATCC #15442), Candida albicans (ATCC #10231), and Aspergillus brasiliensis (ATCC #16404). The assay was performed as described in Example 5, with the modification that fractions of fermentates were used instead of the whole fermentate supernatant. All redissolved fractions were tested at 20% v/v of the media volume in the antimicrobial assays. The readout for the antimicrobial assays was optical density read at 600 nm (OD600). Optical density is a measure of turbidity which is associated with bacterial growth. Fractions with an OD600 less than half of the control (full growth of the organism with no fraction added) for any of the organisms tested against were selected for further evaluation. Individual fractions corresponding to at least 50% inhibition of a target strain at 20% fraction in the growth media, were further analyzed with LCMS analysis.

Results from LCMS, and subsequent MS/MS analysis, identified two main families of antimicrobial actives namely the Brevibacillin Family and the Brevicidine Family. The Brevibacillin Family is closely related to the Bogoral Family of compounds. Table 23 shows the structures of the Brevibacillin variants isolated from the Brevibacillus laterosporus strain A8.11, described herein, compared to a reference Brevibacillin structure (Yang X. et al. 2016. Appl. Environ Microbiol 82:2763-2772). Table 23 shows the structure/sequence of a reference Brevibacillin at the top and then notes any changes in the sequence related to the variant below. If the cell is blank, it has the same sequence as the reference Brevibacillin. The [KY] notation indicates that no b or y ion from MSMS could be found to unambiguously determine the sequence, hence it could be either KY or YK. Table 23 uses the one letter abbreviation of standard amino acids and the three letter abbreviation of non-proteinogenic or non-canonical amino acids. As such, the Dhb refers to dehydrobutyrine, L refers to leucine, Orn refers to Ornithine, I refers to isoleucine, V refers to valine, K refers to lysine, Y refers to tryptophan, M refers to methionine, and M (O) refers to the oxidized version of methionine.

TABLE 23
Brevibacillin Compounds identified in Brevibacillus laterosporus fermentate

| | | | | | | | | | | | | | | |
|---|---|---|---|---|---|---|---|---|---|---|---|---|---|---|
| Brevibacillin | C₆H₁₁O | Dhb | L | Orn | I | I | V | K | V | V | K | Y | L | C₅H₁₃N |

| | | |
|---|---|---|
| Brevibacillin 2V | V | V |

| | | | | | | |
|---|---|---|---|---|---|---|
| Brevibacillin B | | | V | | | [KY] |
| Brevibacillin C | | V | | | | [KY] |
| Brevibacillin D | M | | | | | [KY] |
| Brevibacillin E | | | M | | | [KY] |
| Brevibacillin F | | | | | I | [KY] |
| Brevibacillin G | | | | I | | [KY] |
| Brevibacillin H | M(O) | | | | | [KY] |

| | | | |
|---|---|---|---|
| -Brevibacillin X | V | V | stop |

Tables 23 shows that the Brevibacillus laterosporus fermentate comprised Brevibacillin and nine Brevibacillin variants of which only Brevibacillin (Yang X. et al. 2016. Appl. Environ Microbiol 82:2763-2772) and Brevibacillin 2V (Xinghong Zhao et al., 2021, Front. Microbiol., 17) have been described in literature.

These Brevibacillin variants have at least one lysine residue and Orn, which are cationic. The Orn residues are modified amino acids and are also positively charged. As such, the multifunctional fermentate from Brevibacillus laterosporus can be considered cationic.

The fermentate from Brevibacillus laterosporus also contained some antimicrobial actives identified as Brevicidine variants. Table 24 shows the brevicidine variants isolated from the Brevibacillus laterosporus A8.11 described herein, compared to a Brevicidine reference (Li, Y X. et al., 2018, Nat Commun. 9, 3273).

TABLE 24
Brevicidine Family of actives identified from Brevibacillus laterosporus.

| | | | | | | | | | | | | | |
|---|---|---|---|---|---|---|---|---|---|---|---|---|---|
| Brevicidine | 4-Methyl | Asn | Tyr | Trp | Orn | Orn | Gly | Orn | Trp | Thr* | Ile | Gly | Ser* |
| | Hexanoyl | | | | | | | | | | | | |
| Brevicidine -1 | 4-Methyl | Asn | Tyr | Trp | Orn | Orn | Gly | | | | | | |
| | Hexanoyl | | | | | | | | | | | | |
| Brevicidine -2 | 4-Methyl | Asn | Tyr | Trp | Orn | Orn | Gly | Orn | Trp | Thr | | | |
| | Hexanoyl | | | | | | | | | | | | |
| Brevicidine -3 | 4-Methyl | Asn | Tyr | Trp | Orn | Orn | Gly | Orn | Trp | | | | |
| | Hexanoyl | | | | | | | | | | | | |

The *indicate the residues that are connected in a ring

Table 24 shows that the multifunctional fermentate from Brevibacillus laterosporus comprises Brevicidine variants compounds that have not been described in literature namely Brevicidine-1, Brevicidine-2 and Brevicidine-3.

The Brevicidine family of compounds contain Orn, which are modified amino acids with a positive charge. Due to the presence of the Orn, the overall multifunctional fermentate can be considered cationic.

### Example 10

### Brevibacillus laterosporus Fermentate Supernatant as a Foam Enhancing Agent

Foam enhancing capabilities of the Brevibacillus laterosporus A8.11 fermentate supernatant (cell free supernatant) were evaluated in the presence of different categories of surfactants and detergents commonly found in home and personal care cleaning formulations. To evaluate this, a solution of 1% zwitterionic detergent (CHAPS hydrate), 1% solution of the glycolipid biosurfactant type 1 (acidic sophorolipids non-acetylated), a 0.1% solution of glycolipid biosurfactant type 2 (lactonic (di-acetylated) sophorolipids), and 0.1% of the anionic/non-ionic detergent were each prepared with 10% cell free supernatant in a 50-ml conical tube with deionized water for a final volume of 10-mL. Control samples were prepared individually as 1% zwitterionic detergent, 1% solution of the glycolipid biosurfactant type 1, a 0.1% solution of glycolipid biosurfactant type 2, 0.1% of the anionic/non-ionic detergent and 10% cell free supernatant only in a 50-ml conical tube with deionized water for a final volume of 10-mL. All samples were shaken for 30 seconds by hand and left at room temperature to observe foam height. Foam height was measured by the marking on a 50-ml conical tube and subtracted by 10, where a value of 40 is the maximum foam height and a value of 0 indicates no foam.

TABLE 25-A
Foam height measurements of surfactants and detergents in the presence
of Brevibacillus laterosporus fermentate supernatant over 24 h.

| | | Foam Height | | | | | |
|---|---|---|---|---|---|---|---|
| | | | | Glycolipid | | Glycolipid | |
| | | Zwitterionic Detergent | | Biosurfactant Type-1 | | Biosurfactant Type-2 | |
| | | | (+) 10% | | (+) 10% | | (+) 10% |
| | | Control | cell free | Control | cell free | Control | cell free |
| Time | | (1% only) | supernatant | (1% only) | supernatant | (0.1% only) | supernatant |
| 0 | | 15 | 37.5 | 2.5 | 12.5 | 2.5 | 10 |
| 1 | min | 2.5 | 23 | 1 | 12.5 | 2.5 | 7.5 |
| 5 | min | 0 | 7.5 | 1 | 12 | 2 | 5 |
| 15 | min | 0 | 7.5 | 1 | 12 | 1 | 5 |
| 30 | min | 0 | 7.5 | 1 | 0 | 1 | 2.5 |
| 3 | hours | 0 | 5 | 0 | 0 | 0 | 0 |
| 24 | hours | 0 | 2.5 | 0 | 0 | 0 | 0 |

TABLE 25-B
Foam height measurements of surfactants and detergents in the presence
of Brevibacillus laterosporus fermentate supernatant over 24 h.

Foam Height

Anionic/Non-ionic
Detergent

| | | (+) 10% cell | Cell Free |
|---|---|---|---|
| | Control | free | Supernatant Control |
| Time | (0.1% only) | supernatant | 10% only |
| 0 | 20 | 27.5 | 5 |

| | | | | |
|---|---|---|---|---|
| 1 | min | 20 | 26 | 5 |
| 5 | min | 19 | 25 | 5 |
| 15 | min | 17.5 | 25 | 5 |
| 30 | min | 17.5 | 20 | 4 |
| 3 | hours | 17.5 | 10 | 4 |
| 24 | hours | 12.5 | 0 | 2.5 |

The detergents and surfactants tested in Table 25 A-B show improved foam height in the presence of Brevibacillus laterosporus fermentate supernatant starting at 0 minutes post-shaking compared to controls without the fermentate supernatant, indicating that the fermentate supernatant can serve as a foam enhancing agent in home and personal care formulations.

### Example 11

### Brevibacillus laterosporus Fermentate Supernatant as a Foam Stabilizer

Foam stabilizing capabilities of the Brevibacillus laterosporus A8.11 fermentate supernatant (cell free supernatant) were evaluated in the presence of different categories of surfactants and detergents commonly found in home and personal care cleaning formulations. To evaluate this, a solution of a 1% non-ionic detergent (Tergitol 15-S-7) and a 1% solution of anionic surfactant (Sodium lauroyl sarcosinate) were each prepared with 10% cell free supernatant in a 50-ml conical tube with deionized water for a final volume of 10-mL. Control samples were prepared individually as 1% non-ionic detergent only, 1% anionic surfactant only and 10% cell free supernatant only in 50-ml conical tubes with deionized water for a final volume of 10-mL. All samples were shaken for 30 seconds by hand and left at room temperature to observe foam height. Foam height was measured by the marking on a 50-ml conical tube and subtracted by 10, where a value of 40 is the maximum foam height and a value of 0 indicates no foam. Foam stabilization was defined as the ability to maintain the starting foam height over multiple timepoints.

TABLE 26
Foam height measurements of surfactants and detergents containing
Brevibacillus laterosporus fermentate supernatant

| | | Foam Height | | | | |
|---|---|---|---|---|---|---|
| | | | | | | Cell Free |
| | | Non-ionic Detergent | | Anionic Surfactant | | Supernatant |
| | | Control | (+) 10% | Control | (+) 10% | Control |
| Time | | (1% only) | supernatant | (1% only) | supernatant | 10% only |
| 0 | | 37.5 | 40 | 40 | 40 | 5 |
| 1 | min | 37.5 | 40 | 35 | 40 | 5 |
| 5 | min | 36 | 40 | 12.5 | 40 | 5 |
| 15 | min | 22.5 | 40 | 12.5 | 40 | 5 |
| 30 | min | 20 | 40 | 12.5 | 4 | 4 |
| 3 | hours | 14 | 40 | 0 | 2.5 | 4 |
| 24 | hours | 2 | 0 | 0 | 0 | 2.5 |

As shown in Table 26, non-ionic detergents and anionic surfactants containing Brevibacillus laterosporus fermentate supernatant show improved and stable foam height for 3 hours, and at 15 minutes post-shaking compared to controls alone. This indicates that the fermentate supernatant can be utilized as a solution for foam stabilization in home and personal care formulations.

That what is claimed:

1. A composition comprising:
a) an effective amount of a Brevibacillus laterosporus multifunctional fermentate, or fraction thereof; and,
b) one or more preservative,
wherein each of (a) and (b) is present in the range of 0.001% to 5% by weight of the total composition.

2. The composition of claim 1, wherein the multifunctional fermentate or fraction thereof, enhances said preservative efficacy.

3. The composition of claim 1 or claim 2, wherein the multifunctional fermentate, or fraction thereof, has at least two functions selected from the group consisting of an antioxidant, a surfactant, a foam stabilizer, a foam enhancer, a UB blocker, a blue light blocker, an enhancer of one or more preservatives, and any one combination thereof.

4. The composition of claim 1, wherein the composition is a personal care or cosmetic composition further comprising one dermatologically or cosmetically active excipient.

5. The personal care composition of claim 4, wherein said dermatologically or cosmetically active excipient is an anionic excipient and wherein said effective amount of a Brevibacillus laterosporus multifunctional fermentate, or fraction thereof, enhances said preservative efficacy.

6. The personal care composition of claim 4, wherein said dermatologically or cosmetically active excipient is an anionic excipient and wherein said effective amount of a Brevibacillus laterosporus multifunctional fermentate, or fraction thereof, enhances said preservative efficacy and further provide an additional function selected from the group consisting of an antioxidant, a surfactant, a foam stabilizer, a foam enhancer and any one combination thereof.

7. The personal care composition of claim 5, wherein the anionic excipient, is present in the range of 0.001% to 5% by weight of the total composition

8. The personal care composition of claims 4-7, wherein said composition is in the form of a serum, a hydrogel, a cream, an emulsion, a lotion, a gel, an oil, a wipe, an ointment, a semi-solid formulation, or an aerosol spray.

9. The personal care composition of claim 4, wherein the composition is topically applied.

10. The personal care composition of claim 4, wherein the composition is a synergistic personal care composition comprising a combination of an effective amount of a Brevibacillus laterosporus multifunctional fermentate, or fraction thereof and a preservative, wherein said combination provides a synergistic preservation effect in said personal care composition.

11. The composition of claim 1, wherein the preservative is selected from the group consisting of phenoxyethanol, benzoic acid, sorbic acid, citric acid, sodium benzoate, potassium sorbate, phenyl ethyl alcohol, lauryl ethyl arginate and any combination thereof.

12. A method for enhancing the efficacy of a preservative in a composition, the method comprising adding an effective amount of a Brevibacillus laterosporus multifunctional fermentate, or fraction thereof, to a composition comprising a preservative, wherein each of said preservative and said fermentate are present in the range of 0.001% to 10.0% by weight of the total composition, wherein the multifunctional fermentate enhances said preservative efficacy.

13. A method for enhancing the efficacy of a preservative in a composition while simultaneously providing an additional function to said composition, the method comprising adding an effective amount of a Brevibacillus laterosporus multifunctional fermentate, or fraction thereof, to a composition comprising a preservative, wherein each of said preservative and said fermentate are present in the range of 0.001% to 10.0% by weight of the total composition, wherein the multifunctional fermentate enhances said preservative efficacy while simultaneously providing an additional function selected form the group consisting of an antioxidant agent, a surfactant, a foam stabilizer, a foam enhancer, a UB blocker, a blue light blocker, or any one combination thereof.

14. The method of claim 12, wherein the preservative is selected from the group consisting of phenoxyethanol, benzoic acid, sorbic acid, citric acid, sodium benzoate, potassium sorbate, phenyl ethyl alcohol, lauryl ethyl arginate and any combination thereof.

15. The method of claim 12, wherein the composition is a personal care or cosmetic composition.

16. The method of claim 15, wherein said personal care composition is in the form of a serum, a hydrogel, a cream, an emulsion, a lotion, a gel, an oil, a wipe, an ointment, a semi-solid formulation, or an aerosol spray.

17. The method of claim 11, wherein the composition is topically applied.

18. Use of an effective amount of a Brevibacillus laterosporus multifunctional fermentate in a personal care formulation, wherein said multifunctional fermentate has at least two functions selected from the group consisting of an antioxidant agent, a surfactant, a foam stabilizer, a foam enhancer, a UB blocker, a blue light blocker, an enhancer of one or more preservatives, or any one combination thereof.
